(19) United States
(12) Patent Application Publication
(10) Pub. No.: US 2017/0081707 A1
(43) Pub. Date: Mar. 23, 2017

(54) MONITORING AND MANAGING A FACILITY MICROBIOME

(71) Applicant: Phylagen, Inc., San Francisco, CA (US)

(72) Inventors: Harrison Dillon, San Francisco, CA (US); James Meadow, San Francisco, CA (US)

(21) Appl. No.: 15/308,971

(22) PCT Filed: May 6, 2015
(86) PCT No.: PCT/US2015/029564

§ 371 Date: Nov. 4, 2016

Related U.S. Application Data
(60) Provisional application No. 61/989,430, filed on May 6, 2014.
(60) Provisional application No. 62/000,425, filed on May 19, 2014.

(51) Int. Cl. C12Q 1/68

(52) U.S. Cl. CPC C12Q 1/689

(57) ABSTRACT

Facilities operations can be conducted more safely, efficiently, and cost-effectively by monitoring changes in the facility microbiome and intervening when those changes indicate the likelihood of a deleterious effect there-from.

### FIELD OF THE INVENTION

The present invention provides methods and materials for monitoring and managing the microbiome of a facility and so relates to the fields of microbiology, molecular biology, indoor air quality, occupant health, and facilities management.

### BACKGROUND OF THE INVENTION

The presence of pathogenic microbes in a facility is known to present health risks to the occupants of the building. There is a growing awareness that that the numbers and types of microbes in a building, sometimes referred to as the “built environment microbiome” (“BEM”), might have a dramatic impact on the occupants of the building and the operations that occur in that building. Unfortunately, however, there are few, if any, useful and efficient methods and tools for monitoring the BEM and taking corrective action to prevent harm to the occupants and operations. This present invention meets this need.

### SUMMARY OF THE INVENTION

In a first aspect, the present invention provides methods for characterizing a facility microbiome, said method comprising: (i) collecting samples from a variety of locations in said facility; (ii) subjecting the samples to DNA sequence analysis; (iii) recording the results of the DNA analysis; (iv) repeating steps (i) to (iii) one or more times; and (v) recording any changes in the analysis over time. Applications of this aspect of the invention generally involve an assessment of the state of an entire building, or a key area of a building, or a set of buildings or key areas, including across buildings, at a particular time or during a particular period of time during which there is some expectation that the microbiome is not undergoing intended change as a result of human action. For example, an assessment might be made to determine if a particular microbe or set of microbes is present in any of those locations on a certain day or during a certain operation or during a certain season.

In a second aspect, the present invention provides methods for correlating a facility microbiome with one or more facility operation parameters, said method comprising (i) characterizing the facility microbiome over a period of time; (ii) characterizing a facility operating parameter over said period of time and comparing it to the characterization of the facility microbiome; and (iii) identifying any changes in said facility microbiome that correlate with changes in the facility operating parameter. Applications of this aspect of the invention generally involve an assessment of the state of an entire building, or a key area of a building, or a set of buildings or key areas, including across buildings, during a particular period of time in which actions thought possible or known to affect the microbiome are being evaluated to determine just that—the effect of the change on the microbiome. For example, an assessment might be made to determine if a particular microbe or set of microbes is present in any of those locations after changing some aspect of building maintenance, including, without limitation, alteration of any heating, ventilation, or air conditioning equipment, including controls and/or components; traffic flow of people or goods in the building; cleaning of the building or anything in it; and the like.

In a third aspect, the present invention provides methods for correlating the facility microbiome with facility operation parameters to identify parameters contributing to a changeable facility condition; and methods for changing a facility condition to alter the facility microbiome to achieve a change in a facility performance indicator.

In a fourth aspect, the present invention provides methods for changing a facility condition to alter a facility microbiome to achieve a desired change in a facility performance indicator, said method comprising (i) correlating the facility microbiome with facility operation parameters to identify changes in the facility microbiome that contribute positively or negatively to a facility operation parameter; (ii) identifying changes in the microbiome that correlate with facility operation parameters that can be prevented or caused by altering a changeable facility condition; and (iii) altering the changeable facility condition by altering the facility microbiome to effectuate the desired change in the facility performance indicator.

### BRIEF DESCRIPTION OF THE FIGURES

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a schematic showing data analytics that can quantify system performance by characterizing a microbiome of a health care built environment in accordance with a representative embodiment of the present invention.

FIG. 2 is a schematic showing data analytics that can quantify system performance by characterizing a microbiome of a food processing built environment in accordance with a representative embodiment of the present invention.

FIG. 3 is a schematic showing data analytics that can quantify system performance by characterizing a microbiome of an office built environment in accordance with a representative embodiment of the present invention.

FIG. 4 is a schematic showing how the invention is applied across several facilities at different locations to improve performance.

FIG. 5 shows three graphs demonstrating the ability of Filter 1 to reduce the number and diversity of bacterial operational taxonomic units (OTUs), as defined below, and is described in more detail in Example 5.

FIG. 6 shows three graphs demonstrating the ability of Filter 1 to reduce the number and diversity of fungal OTUs.

FIG. 7 shows four graphs demonstrating the ability of Filter 1 to reduce the number and diversity of plant pollen OTUs.

### DETAILED DESCRIPTION OF THE INVENTION

The present invention provides methods for characterizing a facility microbiome in a manner that facilitates its correlation with one or more facility operation parameters; methods for correlating the facility microbiome with facility operation parameters to identify parameters contributing to a changeable facility condition; and methods for changing a facility condition to alter the facility microbiome to achieve a change in a facility performance indicator.

### DEFINITIONS

The term “bioburden,” as used herein, refers to the number of colony forming units of microbes living on a surface or in a substrate. The term is most often used in the context of bioburden testing, also known as microbial limit testing, which is performed on pharmaceutical products, medical device products and food products for quality control purposes. Bioburden can also refer to the total amount of living microbial cells per unit area of a surface or unit volume of liquid or air.

The term “food product,” as used herein, refers to any product comprising one or more ingredients or parts that are suitable for human consumption. The term “food product” further refers to any product comprising one or more ingredients or parts that are suitable for animal consumption, such as companion and livestock animals.

The term “built environment,” as used herein, refers to any structure or set of structures constructed as a result of human activity and naturally occurring structures inhabited by humans or animals under human care.

The term “facility,” as used herein, refers to a non-naturally occurring structure. In many embodiments, the facility will provide an area for human activity. Facilities therefore include, without limitation, buildings, and vehicles. Buildings include factories (whether enclosed or not), residential structures and hospitals. Vehicles include airplanes, buses, cars, ships, trucks, and vans. A facility can also be a municipality, such as a city or urban area containing a collection of man-made structures that host a microbiome in different areas such as sewers, water supplies, and air in public areas.

The term “facility microbiome,” as used herein, refers to the type, location and number of microbes present in a facility. Characterization of the type and number of microbes may be inferred from analysis of the nucleic acids present in a facility, as determined by taking samples of material from one or more locations in the facility. A microbiome can be characterized and altered in accordance with the methods of the invention without any specific knowledge of the specific genera and/or species present in the facility or area in a facility to be assessed. For example, a microbiome can be characterized solely with reference to the type of genomic DNA or other nucleic acid sampled from the building.

The term “metagenomics,” as used herein, refers to the study of metagenomes, which is genetic material obtained from environmental samples, including but not limited samples from a built environment, including but not limited to the study of samples of nucleic acid taken from the built environment that may or may not contain intact microbial genomes and the study of relatively small segments of DNA amplified or otherwise derived from nucleic acids in such samples.

The term “microbiome,” as used herein, refers to the microorganisms or potential (to refer to the fact that the presence of the nucleic acid indicates an increased potential for the undesired microbe or activity to be present, but does not actually demonstrate that the activity, such as that of an RNA or protein derived from the DNA, exists) biochemical activities (e.g. antibiotic resistance, metabolic pathway, and the like) present in or on the surface of a designated object, which may be, without limitation, an animal, a facility, a human, or a plant, or in a given space such as the air in a room, or contained within a substance such as water. Microbiome refers to the collective set of microbes (including prokaryotic and eukaryotic microorganisms, and viruses) and/or biochemical activities present in these locations, in terms of both identity and relative abundance.

Proliferation refers to cells undergoing cell division to create more cells, whereas dissemination more generally refers to cells changing location within a facility, such as dissemination via a ventilation system without actually requiring proliferation (which may or may not be occurring).

The term “facility operation parameter,” as used herein, refers to an environmental condition in a facility. Such conditions include, without limitation, air flow, exposed surface composition (carpet, ceiling tiles, paint, upholstery, and fabric of staff clothing), lighting (natural and artificial), temperature, relative humidity, frequency of cleaning, chemicals used for cleaning, surface moisture pH, CO₂ level, O₂ level, CO level, NO₂ level, waste container location and frequency of removal, amount of airborne particulates and particle size distribution, amount of airborne pollen, lighting, facility volume, heating and cooling systems, and human occupancy patterns such as occupant density, occupant traffic patterns and occupant diversity.

The term “facility condition,” as used herein, refers to the state of a facility operation parameter in a building. A facility condition may be changeable and so susceptible to manipulation, or unchangeable. Unchangeable is a relative term that simply indicates that certain parameters may not be altered due to conditions which may be inherent or imposed by human decision (e.g., the number and/or location of doors in a facility may be deemed “unchangeable” for purposes of identifying other parameters that might be changed at lower cost, even though it would be technically feasible to do so).

The term “facility performance indicator,” as used herein, refers to a measurable outcome resulting from the operation of the facility. Examples include the frequency, severity and type of infections of patients in a health care facility, yield of raw agricultural material into processed foods or food ingredients, bioburden of processed foods or food ingredients produced by the facility, percent of human occupants sickened, and shelf life of unprocessed produce, processed foods or food ingredients produced by the facility, sterility of pharmaceuticals and medical devices produced by the facility, employee/occupant sick days, employee/occupant allergies, employee/occupant asthma, employee/occupant reduced lung capacity, and operational continuity of the facility, equipment within the facility, or a particular area of a facility.

The term “operational continuity,” as used herein, refers to the length of time a facility or equipment within a facility can be operated without interruption of normal operations for purposes such as cleaning or sterilization. Operational continuity can be interrupted for routine/planned disruptions such as cleaning or unplanned disruptions, such as termination of commercial operations of a cruise ship due to the occurrence of human illness.

The term “reportable incidents,” as used herein, refers to occurrences that are required by law to be reported to a regulatory authority such as FDA, USDA, CDC, and the like.

As used herein, an operational taxonomic unit (OTU) refers to a nucleic acid sequence that is targeted for identification in a sample, i.e., it is a sequence of a nucleic acid that may be in the sample that will be used to infer information regarding, and so characterize, the microbiome of a BE in accordance with the invention. Thus, those of skill in the art will recognize that OTU, as used herein, can be defined as in phylogeny, where an OTU is the operational definition in DNA sequence of a species or group of species (see “Defining Operational Taxonomic Units Using DNA Barcode Data”, Philos Trans R Soc Lond B Biol Sci 360 (1462): 1935-43 (October 2005)). An OTU can be a commonly used microbial diversity unit (see the article “Surprisingly Extensive Mixed Phylogenetic and Ecological Signals Among Bacterial Operational Taxonomic Units”, March 2013). An OTU suitable for use in the invention can also be a nucleic acid sequence that in essence defines the taxonomic level of sampling selected by the user, which, depending on application, may be an OTU that can uniquely identify individual types of microbes, or may alternatively be an OTU that identifies only collective populations, genera, or species of microbes. An OTU may be a nucleic acid sequence used for species distinction in microbiology, where, typically using rRNA and a percent similarity threshold, scientists use OTUs for classifying microbes. In some embodiments, an OTU is a group of sequences identified from a sample that have at least 96%, at least 97%, or at least 98% nucleotide identity to each other. All organisms containing a sequence from the group are considered the same species for purposes of the analysis.

### Facility Microbiomes

The present invention provides data analysis methodology and data analytics that can quantify system performance by characterizing the microbiome of a built environment (BE) to produce actionable information that enables the owner/operator to optimize system design and operations and so improve system performance.

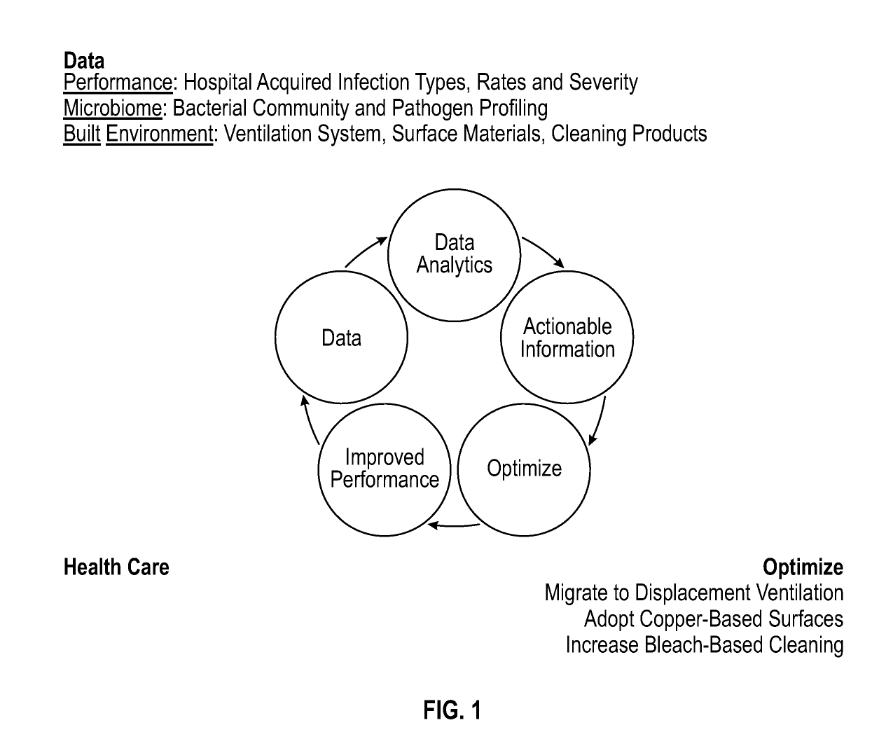

With reference to FIG. 1, a schematic representation is provided which demonstrates various data analytics that may be employed to quantify system performance and characterize a microbiome of a health care BE to improve system performance. In some instances, the performance of a health care BE is characterized through acquiring data regarding various types of performance indicators such as infection types, rates and severity present therein. The microbiome of the heath care BE is further characterized through acquiring data regarding various microbiome indicators, such as the bacterial community and pathogen profile of the facility. Further, the BE of the health care facility may be characterized based on various physical aspects of the facility, such as the ventilation system, surface materials, and cleaning products used. In some instances, the characterization data is used to determine one or more optimization steps that may be employed to improve the performance and characterization of the microbiome of the health care BE. As non-limiting examples, optimization steps may include i) migration to displacement ventilation; ii) adoption of copper-based surfaces; and iii) increased bleach-based cleaning.

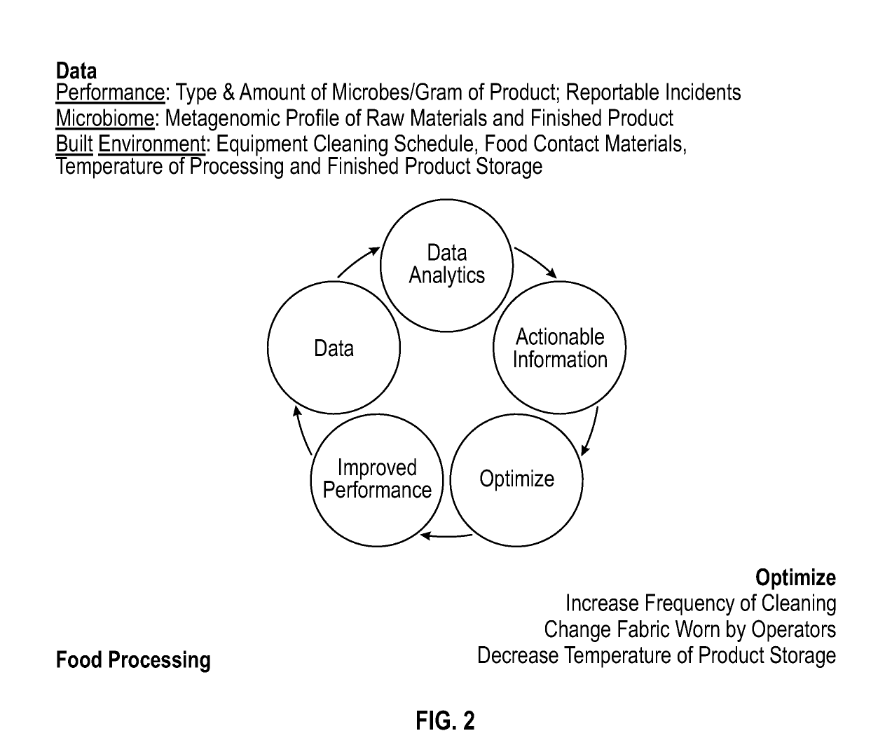

FIG. 2 provides a schematic representation which demonstrates various data analytics that may be employed to quantify system performance and characterize a microbiome of a food processing BE to improve system performance. In some instances, the performance of a food processing BE is characterized though acquiring data regarding various performance indicators, such as type and amount of microbes per gram of product, or reportable accidents. The microbiome of the food processing facility is further characterized through acquiring data regarding various microbiome indicators, such as the metagenomics profile of raw materials and finished products. Further, the BE of the food processing facility may be characterized based on various physical aspects of the facility, such as equipment cleaning schedules, foot contact materials, temperature of processing products, and temperature of finished product storage. In some instances, the characterization data is used to determine one or more optimization steps that may be employed to improve the performance and characterization of the microbiome of the food processing BE. As non-limiting examples, optimization steps may include i) increased frequency of cleaning, ii) change fabric worn by operators; and iii) decrease temperature of product storage.

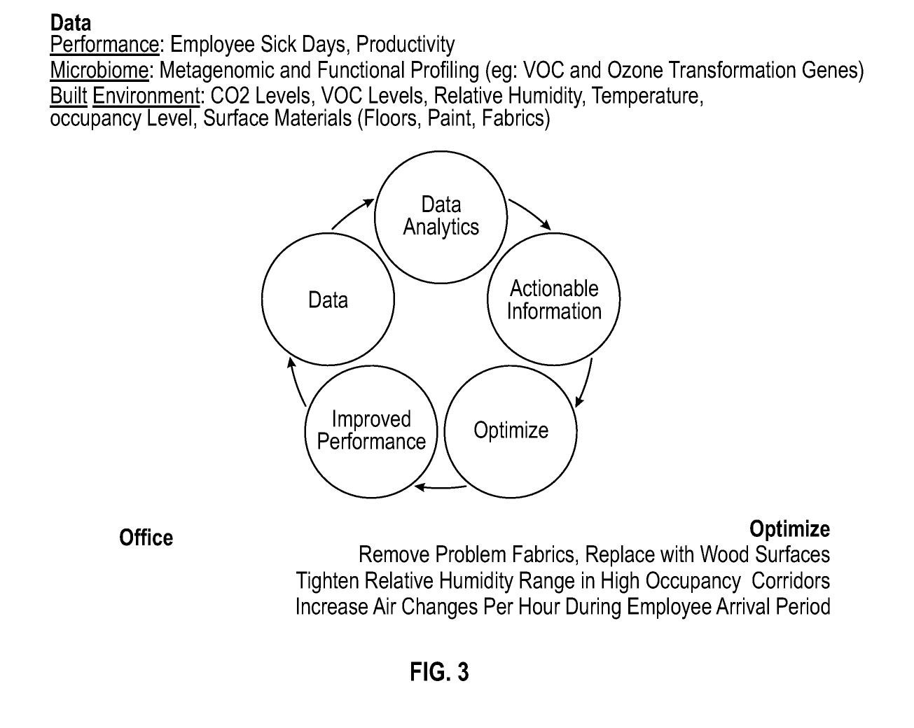

FIG. 3 provides a schematic representation which demonstrates various data analytics that may be employed to quantify system performance and characterize a microbiome of a food processing BE to improve system performance. In some instances, the performance of an office BE is characterized though acquiring data regarding various performance indicators, such as employee sick days and productivity. The microbiome of the office facility is further characterized through acquiring data regarding various microbiome indicators, such as the metagenomics and functional profile of the facility (e.g. VOC and ozone transformation genes). Further, the BE of the office facility may be characterized based on various physical aspects of the facility, such as CO2 levels, VOC levels, relative humidity, temperature, occupancy levels, and surface materials (floors, paint, fabrics, etc.). In some instances, the characterization data is used to determine one or more optimization steps that may be employed to improve the performance and characterization of the microbiome of the office BE. As non-limiting examples, optimization steps may include i) replacing problem fabrics with wood surfaces, ii) tighten relative humidity range in high occupancy corridors; and iii) increase air changes per hour during employee arrival and departure periods.

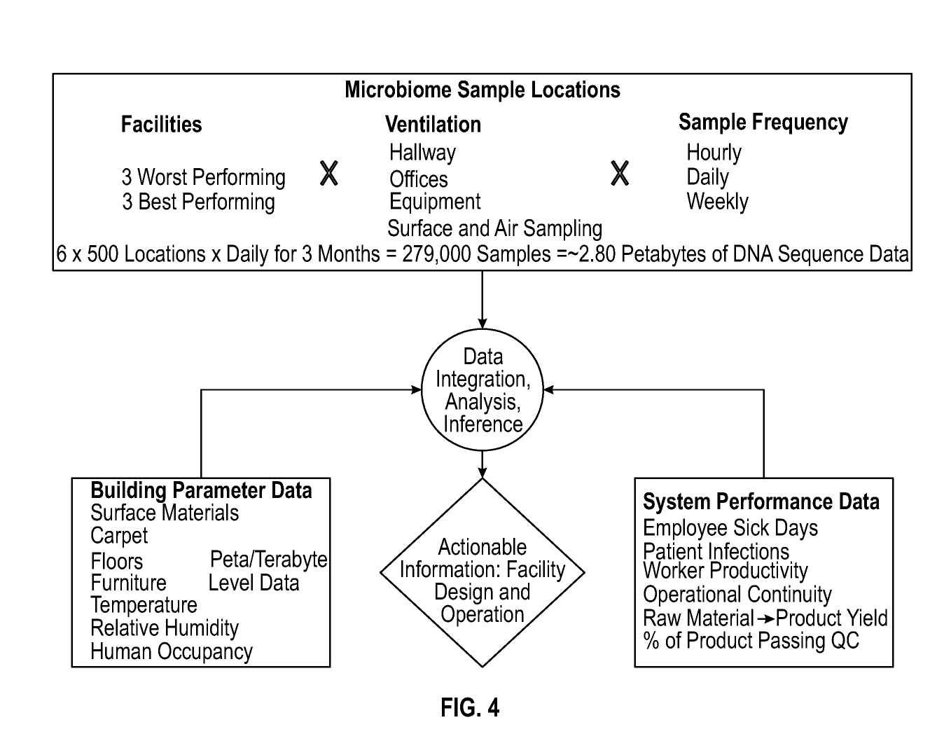

With reference to FIG. 4, a system is shown demonstrating how the present invention may be applied across several facilities at different locations to improve performance for one or more of the facilities.

The BE microbiome (i.e. the collection of micro organisms and/or potential biological activities associated with them in a building or other facility) is influential on occupant health and performance. For example, despite cleaning practices aimed at sterility, all exposed surfaces inside a hospital are covered in countless bacteria and fungi. These are generally dispersed into the building from occupants (including sick patients), ventilation systems, open doors and windows, and materials brought into the building. Modern hospitals are designed to exclude unfiltered outdoor air, however this results in concentrated levels of human-associated microbes indoors, including pathogens and other problematic microbes.

Additionally, cleaning practices (with antibacterial products, for instance) can result in the rapid evolution of antibacterial-resistance genes in bacteria and fungi, and this is especially the case in hospitals. Less problematic microbes, such as those from plants and soils that circulate in outdoor air, can effectively be introduced in ventilated air, which effectively dilutes high concentrations of human-associated microbes. Some microbes are only able to infect a human if present at a concentration above a certain threshold, and changing one or more facility parameters to dilute the concentration of such a pathogen can cause the concentration of the pathogen to drop below the threshold. The dilution can occur through multiple independent mechanisms.

Food processing facilities are highly regulated to avoid proliferation of food-borne, illness-causing organisms. However, just like in hospitals, these facilities are habitually treated with antimicrobial compounds such as triclosan that can unintentionally concentrate antibiotic-resistant organisms.

Office buildings ventilate large quantities of air to maintain occupant comfort. Ventilation design and operation, as well as occupant behavior, can strongly influence the microbes in the air, on surfaces, and those that collect in dust. Current ventilation and design practices are aimed at reducing energy consumption and in particular maintaining occupant thermal comfort, but there are no convenient or practical methods or systems to take indoor microbial content into account, much less characterize it and correlate it with other operational parameters. This can be especially problematic when an airborne- or surfaceborne-disease, such as the flu or measles, is introduced into an occupied office building, and there is no way to detect its presence in the building before many people are infected and corrective and/or ameliorative action taken.

Housing contains many sources of microbes, including people, pets, plants, food, and restrooms. Microbes in houses can have a profound influence on the early-childhood development of disorders like asthma or allergies. Green space near a house can cause a decrease in the risk of asthma and allergies, as is the presence of a dog in the house (and the beneficial microbes they shed inside the house).

There are a variety of design and operation changes that can influence the built environment (BE) microbiome. For example, ventilation plays a key role in influencing the BE microbiome. Indoor and outdoor air can contain drastically different microbial communities, especially in heavily occupied buildings. Introducing unfiltered outdoor air into a building can change the indoor microbiome in a matter of minutes, as can altering the overall porosity and/or selectivity of a ventilation system through bypassing certain filters. For example, during times of day when particulate matter is high, such as during rush hour traffic, a building ventilation system can be operated under high selectivity to eliminate or reduce the particulates above a certain size. During other times, such as at night when nearby roads are relatively empty, the building ventilation system can be operated under lower selectivity to allow a higher proportion of outdoor microbes to enter the building.

Surface materials also play a key role in influencing the BE microbiome. Because humans touch surfaces in the BE, human microbes are ubiquitous on indoor surfaces. Material choices, for instance hard floors versus carpets, and stainless steel versus fabric, change the indoor microbiome. Antimicrobial compounds such as triclosan are embedded in numerous indoor surfaces, such as cutting boards, children's toys, and shower curtains. As a result, these compounds are ubiquitous in indoor dust, and can drive the rapid evolution of antimicrobial resistance, which is ultimately consequential for treating microbial problems.

Another key contributing factor to the BE microbiome is occupant behavior. Movement in the BE resuspends settled dust, and the microbes in dust and on surfaces. Airborne microbes can interact with humans by causing allergic reactions, settling on exposed food, being breathed into lungs, etc.

Building design can also influence the BE microbiome. For example, the proximity of rooms influences microbes present. In other words, adjacent rooms tend to share more microbes than rooms distant from one another. Restrooms are covered in human-associated, and especially human fecal-associated microorganisms. Flushing toilets can aerosolize millions of bacterial cells, which are readily detected in restroom air. Thus rooms adjacent to restrooms are likely to share air containing fecal bacteria.

There are a variety of factors that contribute to the overall condition of the BE microbiome. For example, proliferation of pathogens on indoor surfaces/materials as a result of insufficient cleaning and poor material choices, dispersal of pathogens and allergens (including pollen) from outside due to too much outdoor air at the wrong time, dispersal of pathogens from occupants as a result of lack of effective ventilation, proliferation of allergens in building materials as a result of poor building conditions, excessive moisture, poor ventilation, materials that foster colonization by pathogenic microbes, temperature and relative humidity extremes, excessive human-associated airborne microbes caused by a lack of ventilation during occupation, excessive human-fecal bacteria on surfaces and in air as a result of insufficient cleaning, poor ventilation, and poor placement of adjacent rooms, and lack of beneficial microbiome resulting from poor ventilation, wrong materials, excessive cleaning, and lack of appropriate outdoor air sources.

Accordingly, the condition of a BE microbiome is an important consideration to any facility that experience financial and/or health loss due to indoor microbial problems. Non-limiting examples of challenges which may be presented by poor BE microbiome condition include facility shutdowns, revenue loss, product recalls, product spoilage, productivity loss from unwell employees or occupants, airborne outbreaks (flu, measles, and other diseases caused by microbes), asthma, allergies (both triggers and causes) and other forms of reduced respiratory function, hospital acquired infections (MRSA, C. difficile, etc.), mold contamination, and occupant discomfort. Some embodiments of the present invention provide for improved BE microbiome conditions as manifested by the following non-limiting indications: detection of fewer targeted pathogens/allergens, fewer HAIs, reduced volatile organic compounds, reduced odor; outbreak stop/avoidance; improved occupant comfort; service/product/facility continuity; and improved indoor air quality.

### Types of Facilities and Performance Indicators

Health Care Facilities

The present invention has application in health care facilities such as hospitals, surgery centers, and dialysis centers. Facility performance indicators typically include at least one of type, severity and frequency of human or animal infections experienced, detected, and/or measured within the facility. Non-limiting examples of microbes and infection types and biochemical activities that cause or can cause reductions in performance include ventilator-associated pneumonia, Staphylococcus aureus (including methicillin resistant strains), Candida albicans, Pseudomonas aeruginosa, Acinetobacter baumannii, Stenotrophomonas maltophilia, E. coli O157:H7, Clostridium difficile, Tuberculosis, Urinary tract infections, pneumonia, Gastroenteritis, Enterococcus (including Vancomycin-resistant strains), Legionnaires' disease, Puerperal fever, antibiotic resistance, specific metabolic pathways or enzymes in them, and specific types of genes or gene segments.

Factory Facilities

The present invention further has application in factory facilities including food processing and manufacturing plants in which raw or partially processed food is converted into a further processed food or a finished food ready for packaging. Non-limiting examples of factory facilities include plants or business where one or more of the following processes take place: yogurt production, poultry processing, ground beef production, vegetable processing (lettuce/ready-to-eat salads, carrots, tomatoes), and nuts/peanut butter production. Microbial contamination within a factory facility generally leads to a reduction in performance, which causes loss of product, and increased wastage. Non-limiting examples of performance reducing microbial contamination, and related illnesses, include E. coli, including O157:H7, botulism, bovine spongiform encephalopathy, Listeria, Campylobacter, norovirus, Trichinosis, Staphylococcus aureus, and Salmonella, and the genes and biochemical activities uniquely or specifically associated with them.

Livestock rendering plants are food processing factory facilities where animals, such as pigs and cows, are slaughtered, cleaned, and/or cut into usable portions for either sale directly to consumers or use by an additional processing facility to make finished products such as sausage, ground beef, and the like. These types of factory facilities are also susceptible to microbial contaminations and reductions in performance, as described herein.

Breweries and wineries are beverage processing factory facilities that perform controlled microbial fermentation to manufacture beer, wine, distilled spirits, and/or herbal or tea-based drinks such as kombucha. These types of factory facilities are also susceptible to microbial contaminations and reductions in performance. For example, in some instances microbial contaminations result in the production of product that fails to meet desired or legally required specifications. These batches are thus unusable and result in lost profits. Non-limiting examples of performance reducing microbes include those identified above, as well as naturally occurring microbes present within the produced food or beverage.

Other food processing factory facilities include dairy and non-dairy farms. Dairy farms generally include farms where milk is collected from milk-producing animals, such as cows, goats, and sheep. Non-dairy farms generally include barns where livestock is kept in cages and shelters, such as chicken houses. These types of factory facilities are replete with all different types of microbes that must be monitored and controlled to prevent microbial contamination leading to performance reduction. For example, avian influenza is a major problem in chicken houses.

Non-dairy farms may also include fish farms, such as freshwater and saltwater facilities that cultivate various varieties of fish (such as salmon, trout and tilapia) and shellfish (such as clams, oysters, mussels, shrimp, lobster, and crabs). Many fish farms have water conditioning devices which utilize a fixed bed substrate that harbors microbes that facilitate health of the fish. This fixed bed substrate commonly harbors such desirable microbes. Performance improvement may occur as water for the tanks and pools is run through the water conditioning device, thereby exposing the tanks and pools to microbes that have proliferated in the device.

Factory facilities may further include facilities or plants in which pharmaceutical manufacturing is performed. These types of facilities include those that manufacture drugs intended to treat or cure disease, as well as those that produce over-the-counter medications, such as aspirin and acetaminophen. Pharmaceutical manufacturing facilities may be either biological-based (CHO, E. coli) or synthetic chemistry-based. In either case, these types of facilities are susceptible to performance reducing microbial contamination, as discussed herein.

Medical device manufacturing factory facilities are facilities or plants in which devices are manufactured for the purpose of treating and/or curing a medical condition. Some medical device manufacturing facilities provide invasive medical devices, such as syringes, catheters, artificial joints, and pacemakers. As will be readily appreciated by those of skill in the art, these types of factory facilities and medical devices require utmost attention in preventing microbial contamination, and therefore will benefit from practices of the present invention.

### Vehicles

The present invention may be implemented within any compatible vehicle. A non-limiting example of a vehicle contemplated by the instant invention includes a cruise liner. Cruise liners are ships used primarily for recreation, particularly those which house more than 100 people for multiday trips. Other non-limiting examples of vehicles include submarines and commercial aircraft. The close and contained environment within these types of vehicles commonly leads to microbial contamination and thus performance reduction. Non-limiting examples of performance reduction for these vehicles includes various illnesses caused by bacteria and viruses, particularly norovirus, suffered by passengers and/or crew.

### Housing

The present invention further has application to any type of human housing, but will have particular benefit for structures that accommodate large numbers of people, such as prisons, retirement and assisted living homes, hotels, hospitals, doctor offices, medical centers, athletic facilities and gyms, public pools, public bathrooms, schools, and dormitories. In particular, facilities that house humans with reduced immune function benefit from various embodiments of the invention.

### Consumer Food Facilities

The present invention has application to any type of consumer food facility, including restaurants and retail grocery stores. In one embodiment the restaurant is part of a chain (2 or more locations) that has standardized facilities and equipment between locations. Equipment for soring perishable food, such as cold cases for meats, seafood, refrigerated liquids such as milk, and produce is commonly found in these facilities.

### Equipment

The present invention has application to any type of equipment in any factory or health care facility, such as, for example and without limitation cell phones, desktop and portable computers, keyboards, staff badges, meat slicers, extruders, fermenters, mixers, culinary machinery and devices, surgical instruments, door knobs, glassware, medical devices, and various types of wheeled equipment.

### Microbiome Sampling

Types of Samples

In accordance with the invention, a characterization of the microbiome of a facility will be determined from samples obtained from the facility. Suitable sources of samples include air, dust, surface materials, and water. Samples are collected for the analysis of the nucleic acid (DNA or RNA) in them and so are collected and processed in a manner intended to minimize degradation of the desired nucleic acid intended for analysis.

Frequency of Sampling

In accordance with the invention, the microbiome of a facility is characterized at a point in time or during a period of time or monitored for changes over time or monitored with changes intended to alter the microbiome being implemented contemporaneously. In some instances, the microbiome is monitored for a period of time lasting from minutes to more than a year. In one embodiment, a microbiome is monitored for 24 hours. In one embodiment, a microbiome is monitored for three to seven days. In one embodiment, a microbiome is monitored for one to three months. In one embodiment, a microbiome is monitored for an extended period of time, such as for a period exceeding a year. In some instances, a microbiome is monitored for the life of a facility comprising the microbiome. In some embodiments, the microbiome is assessed only once or only once during some periodic cycle (months, years)

In some instances, change in the facility's microbiome is determined by comparing results from multiple samples obtained during a sampling period from the same facility (same or different locations) or from similar or selected diverse facilities. In many embodiments, samples are collected two or more times over the course of a sampling period. The frequency of sample collection may be hourly, daily, monthly, yearly, or any combination thereof and will vary depending on the facility and the intended purpose of the monitoring.

### Methods of Sampling

Samples may be obtained by any means that does not materially alter or destroy the target molecules contained therein. Target molecules may comprise any biological material of interest, including, but not limited to microbes, viruses, DNA, RNA, proteins, spores, bacteria, pathogens, microbial VOCs, or any chemical product of microbial metabolism.

Various sampling methods may be used to collect target molecules. In some instances, a sampling method is selected based upon the specific characteristics of the facility from which the target molecules are being collected. For example, in hospitals, office buildings, and schools non-disruptive sampling is preferred, and thus necessitates quiet vacuum pump air collection or passive sampling methods. More disruptive sampling methods, such as high-volume vacuum pump air collection, are more appropriate in manufacturing facilities where excessive noise is acceptable.

In some instances, a sampling method is selected based upon desired data or analysis parameters. For example, tracking known pathogens on hospital surfaces can be accomplished by collecting surface swabs, while monitoring airborne microbiome dynamics in an office environment requires air sampling, which may be continuous or intermittent, depending on the application. As another example, identifying allergens in the airborne microbiome requires collecting dry microbes, as on a dry vacuum filter, because microbial viability is not necessary for allergenicity. On the other hand, collecting data on live pathogens in an operating room requires information about microbial viability, and thus collection, at least for certain embodiments, must preserve cells in their current form, as in a preservative liquid. Additionally, when indoor air quality is being considered, simultaneous collection of non-biological environmental parameters may be important, such as particulate matter, VOC concentration and content.

Surface Sampling Methods

Surfaces can be sampled to assess the quality, type, identity, metabolic profile, allergenicity, and gene content of various target molecules, such as, for instance, microbial cells. Surface samples may be obtained from any surface having a surface area of sufficient size from which to collect the sample. For example, in some instances a surface sample area may range from 1 cm², to a tabletop surface, to an entire facility floor or wall. Determination and selection of surface sample area is largely driven by the desired data or analysis parameters for a given microbiome or facility. For example, monitoring pathogens on the surfaces in an operating room will require sampling a variety of surfaces and a range of surface sizes throughout the facility, including door handles, instruments, table tops, walls, and electronic devices. Determination and selection of surface sample area may also be driven by the amount of available biomass on the selected surface (i.e. quantity of target molecules).

Suitable surfaces for sample collection may include any solid or semi-solid surface that is accessible for sampling. For example, suitable surfaces include vertical surfaces, horizontal surfaces, textured surfaces, smooth surfaces, wetted surfaces, dry surfaces, human skin, hair, plant leaves, HVAC filters, ventilation systems, door handles, outdoor surfaces, fabrics, and so forth.

Surface sampling is often done by swabbing a selected surface with a sterile cotton or nylon swab. In some instances, the sampling is done with a dry swab. In other instances, the sampling is done with a swab that has been wetted with a sterile, stabilizing buffer solution. Buffer solution is generally selected based upon the biological needs or other characteristics of the target molecules. For example, when microbial-function data are of interest, a buffer that preserves RNA is most appropriate, whereas preservation of DNA will require a different type of buffer liquid.

The buffer can help dislodge target molecules from the selected surface and attract the target molecules onto the swab bristles or the wipe fibers. The buffer further acts to stabilize the microbial activity, if any, of the target molecules. In some instances, a sterile cotton or nylon wipe is used in place of a swab.

Material picked up from the selected surface is typically rinsed from the swab with sterile solution. In some instances, the sterile solution comprises a buffer solution used during collection of the surface sample. Surface samples are immediately stored in sterile containers, frozen, and transported to a freezer facility until laboratory processing. As such, the target molecules are preserved from degradation.

Nucleic acids are then extracted and subjected to various sequencing methodologies which may or may not include fragmentation, cloning and amplification. For air and other gases, sampling may be done, for example and without limitation, using a vacuum pump to pull the air or other gas through a filter to which microbes adhere or become otherwise entrapped. Water/liquid samples can also be obtained from sources such as drains.

Air Sampling Methods

Air can be sampled to assess the quality, type, identity, metabolic profile, allergenicity, and gene content of various target molecules. Air samples may be obtained via various well known techniques in the art, including but not limited to passive settling dish assays (empty, sterile petri plate), passive static-charged cloth assays, and vacuum air pump collection using at least one of a sterile button filter (such as SKC celllose membrane filters), a sterile filter cup (such as Nalgene Polypropylene Analytical Test Filter Funnel), and a liquid impinge (such as SKC BioSampler).

Passive Air Sampling

Passive samplers can be used to collect particles and bioaerosols that settle out during the sampling period. Passive samplers are generally inexpensive and thereby greatly reduce the cost and the need of infrastructure in a facility or on the sampling location. Passive samplers are semi-disposable and, due to their low cost, can be employed in great numbers, allowing for a better cover and more data being collected. In some instances, passive samples are small in size and thereby may be easily the passive sampler can also be hidden, and thereby lower the risk of disturbance. Non-limiting examples of passive sampling devices include sterile petri plates, diffusive gradients in thin films (e.g. DGT samplers), Chemcatcher, Polar organic chemical integrative sampler (POCIS), and air sampling pumps.

Following the sampling period, the passive samplers are collected and the target molecules are collected from the sampling devices. In some instances, the target molecule samples are collected by swabbing one or more surfaces of the passive samplers. In other instances, the target molecules are collected by washing one or more surfaces of the passive sampler with a small volume of liquid buffer solution. The collected samples are then suspended in a buffer solution until further laboratory processing.

Static-Charged Cloth Air Sampling

Static-charged cloths collect target molecules, including cells and bioaerosols, by static attraction. Following the sampling period, the target molecules are extracted from the static-charged cloths by i) dissolving the static-charged cloth in a buffer solution; ii) washing the static-charged cloth in buffer solution; or iii) washing the static-charged cloth in a charged buffer solution to release the target molecules from the cloth.

Vacuum Drawn Air Sampling

Vacuum drawn air samplers typically include a porous air filter coupled to a vacuum air pump. Air is drawn through the filter and target molecules larger than the pore size of the filter settle on the filter. Filter pore size may vary depending upon the desired target molecule. In some instances, a vacuum drawn air sampler is selected having a filter pore size from 0.2 um diameter to 5 um diameter, wherein the vacuum drawn air sampler is used to collect target molecules selected from the group consisting of bacterial cells, fungal cells,

Liquid Impinger Sampling

A liquid impinger sampler is a device in which target molecules are removed from air by impacting the target molecules into a liquid. Liquid impinger air samplers capture target molecules in liquid, such as a buffer solution, water, or stabilizing buffer solution. Vacuum-drawn air is pulled through a liquid medium, thereby trapping the target molecules in the liquid. Liquid impinger samplers are most useful for counting cells, capturing live cells, and capturing viable molecules, such as DNA, RNA and proteins.

Microbiome Sample Analysis

Once samples are obtained, they are analyzed in accordance with the instant invention to provide a characterization of the microbiome. The characterization typically involves identification of nucleic acids in the sample by sequence analysis. Alternatively or additionally, collected target molecules may be used to count cells, i.e., to infer the number of cells present, and, as noted above, whole cells can be collected, and if collected to ensure viability maintained, even to grow viable cells in culture.

Typically, however, the target biological material will be a nucleic acid molecule, and the sample will be subjected to a process to extract nucleic acid, which may include DNA or RNA, for sequence analysis. Sequence analysis may be performed in accordance with various embodiments of the invention by determining the nucleotide sequence of all nucleic acid in the sample or by some portion thereof. In some embodiments, sequence analysis may be determined by hybridization to a probe or an array of probes, including probes immobilized on a microarray. In other embodiments, sequence analysis is performed by nucleic acid sequencing. Sequencing of RNA can be used as an indicator of viability of the cells and so to determine cell viability at the time of sampling, as well as determining which biochemical activities are present at the sample location (as opposed to taxonomic determination).

In some embodiments in which only a subset of the nucleic acids in a sample are characterized, the sequence analysis may be targeted to specific DNA or RNA sequences, such as those associated with 23S rRNA or 16S rRNA, which can be used to identify which species/genus/taxa of microbes are present and the relative abundance of each; those associated with antibiotic resistance genes (see Liu and Pop. ARDB-Antibiotic Resistance Genes Database. Nucleic Acids Res. 2009 January; 37 (Database issue): D443-7); or those associated with indicator genes, which are genes associated with improved performance of the system or reduced performance of the system and which may or may not have a known function. In some embodiments, the antibiotic resistance genes or other indicator genes themselves are sequenced, either as part of a metagenomic sequencing or as amplified products. In some embodiments, the sequence identification step will involve the determination of whether any nucleic acid sequences associated with an indicator taxa is present. An “indicator taxa” is an organism that is associated with a positive or negative impact on a performance indicator. For example, MRSA is a bacterial indicator taxa for many facilities, as are other pathogenic organisms. An overabundance or underabundance of an indicator taxa, or genes associated with an indicator taxa, within a microbiome may be used to determine current and/or future under, or over-performance of the system. An indicator taxa can also be an OTU or a subset of an OTU.

In some instances, an indicator taxa or OTU is antibiotic resistant. Antibiotic resistance is a form of drug resistance whereby at least some sub-populations of a microorganism are able to survive after exposure to one or more antibiotics. In some instances, an indicator taxa is resistant to multiple antibiotics and is considered multidrug resistant; such organisms are sometimes more commonly referred to as superbugs.

Antibiotic resistance may take the form of a spontaneous or induced genetic mutation, or the acquisition of resistance genes from other bacterial species by horizontal gene transfer via conjugation, transduction, or transformation. Many antibiotic resistance genes reside on transmissible plasmids, facilitating their transfer. Exposure to an antibiotic naturally selects for the survival of the organism with the genes for resistance. In this way, a gene for antibiotic resistance may readily spread through a microbiome.

In the simplest instances, antibiotic resistant indicator taxa have acquired resistance to first-line antibiotics, thereby necessitating the use of second-line agents. In the case of multidrug resistant indicator taxa, resistance to second- and even third-line antibiotics is acquired. For these types of indicator taxa or OTUs, timely detection and monitoring of the microbiome may be important to prevent performance reduction.

Non-limiting examples of antibiotic resistant indicator taxa include Staphylococcus aureus, methicillin-resistant Staphylococcus aureus, Pseudomonas aeruginosa, Klebsiella pneumonia, Mycobacterium tuberculosis, Neisseria gonorrhoeae, vancomycin-intermediate S. aureus, vancomycin-resistant S. aureus, extended spectrum beta-lactamase, vancomycin-resistant Enterococcus, fluoroquinolone-resistant Salmonella, fluoroquinolone-resistant E. coli, clindamycin-resistant C. difficile, and multidrug-resistanct A. baumannii.

In some embodiments, metagenomics is performed, such that all of the DNA (or all of the nucleic acid or all of the RNA) from a sample is sequenced. This may be done with or without an amplification step, but in many instances, there will be no amplification step. This provides information about not only which species/genus/taxa of microbe is present and its relative abundance, but also which genes, known or unknown, are present. For example, genes encoding biochemical activity such as antibiotic resistance, production or destruction of volatile organic compounds, allergenicity, toxins, and other indicator genes are present in the sample. Below in Table 1 is a list of exemplary antibiotic resistance genes that can be used as part of a reference database. Sequences identified in a microbiome sample are checked for identity comparison to these predetermined sequences. Levels of identity of 80% or higher are generally considered by those skilled in the art to be indicative of a sequence encoding the same or similar biochemical function. Algorithms for analyzing raw sequence data from samples can set the desired level of identity, such as greater than 50%, greater than 60%, greater than 70%, greater than 80%, or greater than 90% identity to any one of a set of predetermined sequences in a reference database.

TABLE 1
List of Exemplary Antibiotic Resistance Genes

| GenBank accession | SEQ ID | | | |
|---|---|---|---|---|
| number | NO | Organism | Type of gene | Mechanism |
| X63598.1 | SEQ ID | Staphylococcus | mecR1/mecI | Methicillin-resistance regulatory |
| | NO: 1 | aureus | | protein for mecA |
| ABWO01000112.1 | SEQ ID | Tyzzerella | Class A beta- | Enzyme opens the beta-lactam |
| | NO: 2 | nexilis | lactamase | antibiotic ring |
| NZ_ABBK01000507.1 | SEQ ID | Burkhoderia | Resistance- | Multidrug resistance efflux pump |
| | NO: 3 | pseudomallei | nodulation-cell | |
| | | | division | |
| | | | transporter | |
| | | | system | |
| DQ061191.1 | SEQ ID | Pseudomonas | Class B beta- | Enzyme opens the beta-lactam |
| | NO: 4 | aeruginosa | lactamase | antibiotic ring |
| DQ141319.1 | SEQ ID | Klebsiella | Class D beta- | Enzyme opens the beta-lactam |
| | NO: 5 | pneumoniae | lactamase | antibiotic ring |
| DQ141318.1 | SEQ ID | Acinetobacter | Group A drug- | Cannot be inhibited by |
| | NO: 6 | baumannii | insensitive | trimethoprim |
| | | | dihydrofolate | |
| | | | reductase | |

Genes encoding enzymes involved in volatile organic compound degradation (an example of a metabolic pathway) are known and can be used in a reference database: for example see Applied Biochemistry and Microbiology 5-2005, Volume 41, Issue 3, pp 259-263 “Metabolic pathways responsible for consumption of aromatic hydrocarbons by microbial associations: Molecular-genetic characterization” Khomenkov et al.

The ability to modify the way a facility is operated in response to biochemical activities detected in the genes of microbes in the built environment is an object of the invention. Traditional methods of building hygiene involve cleaning surfaces and filtering air without knowing what microbes and biochemical activities are being eliminated. Conventional wisdom has long been that microbes (viruses, bacteria, and fungi) are simply undesirable pollutants that should be reduced or eliminated indoors. The revelation that over 90% of the cells in a healthy human body are microbes demonstrates that humans have co-evolved with microbes and a “scorched earth” attempt at elimination of microbes from the environment removes both pathogens as well as microbes that are beneficial to the human body. Promotion of beneficial microbes is just as important as elimination of undesirable ones in a building, particularly considering that humans spend 90% of their lives indoors. The genetic composition of microbiome samples from a BE can reveal all or key components of the possible biochemical activity from the microbes present, not just which microbes are there (such as from 16S sequencing) or which metabolic activities are active (such as from proteomics or analyzing individual metabolites).

For example, if antibiotic resistant microbes could be a problem in a facility, such as a hospital or assisted living facility, then, in accordance with the present invention, the presence of the activity or a nucleic acid that encodes a protein with such activity, reveals that a type of threat is in the environment, and this may be much more valuable than a test that simply indicates the identity of the presence of a single, specific, undesired pathogen, such as MRSA. Antibiotic resistance activity in a BE is also important in facilities that house animals for livestock production, such as chicken houses and other facilities for poultry production.

In other instances, the products of microbes, such as volatile organic compounds, are a problem inside a BE and identification of the capacity to produce VOCs through identification of genes encoding VOC-producing enzymes may be more useful than identification of a single type of microbe. Some methods of the invention can be used to detect the potential presence of any compound produced by a biochemical pathway in the facility.

In other cases, building standards may require the genes in the BE be known before the building can be certified. Removal or introduction/promotion of one or more biochemical activities in a BE is enabled through methods of the invention.

Some implementations of the instant invention use high-throughput screening technologies and methods to analyze the microbiome of at least one of a facility, a vehicle, housing, a consumer food facility, or equipment. High-throughput screening methods generally use robotics, data processing and control software, liquid handling devices, and sensitive detectors to rapidly conduct high numbers of chemical, genetic, or pharmacological tests. In some instances, high-throughput technologies and methods are capable of screening approximately thousands to millions of samples per hour. In some instance, up to 10 million samples per hour are screened.

In at least one embodiment of the present invention, microbiome samples are collected and initially analyzed via a high-throughput screening system. After a period of time, microbiome samples are again collected and analyzed via the high-throughput screening system. The microbiome sample data is then processed by the high-throughput screening system to detect changes in the microbiome. In some instances, the data processing software of the high-throughput screening system is configured to identify correlations between changes in the microbiome and changes or events in facility operation parameters, as well as changes in facility performance. This data may thus be used to guide facility operation parameters to achieve a desirable microbiome and therefore better performance of the facility.

In some instances, a microbial profile of a microbiome, a form of microbiome characterization, is determined and monitored over time through sampling and DNA typing or profiling. Sampling may be accomplished by any known method in the art. For example, and as described in detail above, in some instances sampling is achieved by swabbing one or more surfaces of the microbiome with sterile cotton swabs. In other instances, sampling is achieved through the use of various sensors strategically placed within the facility and configured to detect or collect one or more indicator taxa. Further, in some instances sampling is achieved through the collection of product samples, water samples, air samples, soil samples and/or biological samples that may comprise one or more indicator taxa. In some instances using mobile sequencing, the sequence data is transmitted to a server location where the sequence data is compared to a reference database.

Under conditions where a certain gene or genetic signature pattern observed by the mobile sequencing device matches a predetermined pattern in the database, an instruction or set of instructions on altering one or more facility operating parameters may be sent to the facility in accordance with the methods of the invention. Examples of instructions are altering the temperature, relative humidity, indoor/outdoor air ratio, amount and type of air filtration (through bypassing or not bypassing certain filter types that filter based on size or organic molecule removal), and indicating a need for additional surface cleaning.

A DNA profile of the microbiome may be obtained by any known method in the art. In some embodiments, microbiome samples are analyzed via one or procedures selected from the group consisting of RFLP analysis, PCR analysis, STR analysis, Illumina sequencing, and AmpFLP analysis. One having skill in the art will appreciate that the DNA profile of the microbiome may be determined by other suitable analytical techniques.

Generally, microbial DNA is extracted from the collected samples and sequenced through various steps of cellular and genetic digestion via the use of detergents, buffers, mechanical disruption, and restriction enzymes. In some instances genetic markers may be used to identify and/or quantify a specific indicator taxa or other type of organism within the sample quickly and accurately. In other instances, a high-throughput screening method is utilized to extract and analyze DNA from the collected samples. In other instances, a high-throughput system is utilized to further perform nucleic acid sequencing of the extracted microbial DNA.

As briefly noted above, mobile DNA sequencers, which can be handheld or wall mounted, can also be used for sequencing facility microbiome samples in many important embodiments of the invention. Currently-available molecular sequencing technology, for example the Oxford Nanopore MinION (Oxford Nanopore Technologies, Oxford, UK), generates thousands of targeted DNA amplicon sequences within 6 hours, including DNA preparation, loading, sequencing, dataset generation, and basic bioinformatic analysis. The device is smaller than an iPhone, and plugs into a laptop computer via USB, and can linked to a wireless or Ethernet connection for sending sequence data. Cloud-based real-time bioinformatic capabilities for field processing and analysis are also possible with the device. One advantage of this technology over current high-throughput sequencing methods is much longer sequence reads that enable species- and/or strain-level identification. Sample preparation for this technology currently includes the following steps: DNA extraction, PCR or fragmentation, end repair and hairpin ligation (to be captured by the MinION pores), incubation. This is the first generation of such near-real-time sequencing technology and anticipated improvements will simply make the methods of the present invention easier and more cost efficient to implement. As an example, a wall-mounted device can be programmed in accordance with the invention to detect a suite of indoor microbial agents or biochemical activities (as determined by comparison of samples to a set of predetermined sequences in a reference database), and then trigger the building's HVAC system to respond when target sequences or sequences with at least a certain amount of sequence identity to a predetermined reference sequence are detected.

Facility Operation Parameters

Any facility operation parameter may be correlated with the facility microbiome and microbiome changes in accordance with the invention. Certain operation indicators will be more typically (commonly) evaluated and are discussed for illustrative purposes below.

HVAC and Ventilation Design

The heating, ventilation, and air conditioning systems (“HVAC”) of a facility constitute key operation parameters that encompass a number of subsidiary operation parameters, such as airflow rate, filtration, and facility filter pore size (as well as the frequency of changing filters, bypassing filters under certain conditions), temperature and temperature fluctuations of the air, indoor/outdoor air ratio entering the HVAC system, and the humidity of the air. Mechanical ventilation and natural ventilation can both be used in a facility. Displacement ventilation can also be used. The number of air changes per hour using a given ventilation system is an example of a facility operation parameter. Some facility operation parameters for certain types of facilities such as hospitals are mandated by law.

Cleaning Regime

The cleaning regime of a facility is another key operation parameter that encompasses a number of subsidiary operation parameters, such as the chemicals used, the surfaces cleaned, the method of cleaning, and the frequency of cleaning. Sterilization procedures (for hospitals in particular, which often use UV light and/or chemicals to clean) also represent key operation parameters.

Surfaces Present

The type of surfaces (e.g. carpet versus hard floor and composition, e.g., fiber, wood, linoleum) present in a facility constitute key operation parameters. All surfaces (ceiling, floors, walls, doors and doorknobs, equipment surfaces, and the like) in a facility, their location(s) and their relative abundance constitute operation parameters useful in accordance with the methods of the invention.

Facility Performance Indicators

Any facility performance indicator may be correlated with the facility microbiome and microbiome changes in accordance with the invention. Certain facility performance indicators will be more typically evaluated and are discussed for illustrative purposes below.

Rate of Infection/Sickness/Mortality

The frequency, severity and type of infections, sickness, and/or mortality of the occupants (human and/or animal), as well as the outcome of any treatment of infection or sickness, are key facility performance indicators. Employee sick days/absenteeism is another performance indicator that can be related to sickness as a result of a facility microbiome. Lung function and all subsidiary measurements of lung function of facility occupants is a facility performance indicator. For example, average lung capacity of employees in a facility can be measured, and facility operation parameters can be altered in response to the presence of microbiome signatures that have been observed in the past (in that facility or others) to cause reduced lung function.

There is a general concern in industry today that the built environment microbiome (BE) can negatively or positively impact employee health and so impact profits and performance, and air quality is a key parameter in the BE. Reduced lung capacity is a major employee health concern and can occur through poor BE air quality. For example, a variety of microbe-induced mechanisms, and therefore a variety of microbiomes, can affect the respiratory system directly or indirectly. Lung function can be measured through a spirometry device to generate a pneumotachograph. Relevant spirometry measurements include includes tests of pulmonary mechanics which include measurements of FVC (forced vital capacity: the determination of the vital capacity from a maximally forced expiratory effort), FEV₁ (volume that has been exhaled at the end of the first second of forced expiration), FEF values (FEFₓ: forced expiratory flow related to some portion of the FVC curve; modifiers refer to amount of FVC already exhaled; FEFₘₐₓ: the maximum instantaneous flow achieved during a FVC maneuver), and forced inspiratory flow rates (FIFs: Specific measurement of the forced inspiratory curve is denoted by nomenclature analogous to that for the forced expiratory curve). For example, maximum inspiratory flow is denoted FIFₘₐₓ. Unless otherwise specified, volume qualifiers indicate the volume inspired from RV at the point of measurement), MVV (maximal voluntary ventilation: volume of air expired in a specified period during repetitive maximal effort), tidal volume (VT: that volume of air moved into or out of the lungs during quiet breathing), inspiratory reserve volume (IRV: the maximal volume that can be inhaled from the end-inspiratory level), expiratory reserve volume (ERV: the maximal volume of air that can be exhaled from the end-expiratory position), residual volume (RV: the volume of air remaining in the lungs after a maximal exhalation) total lung capacity (TLC: the volume in the lungs at maximal inflation, the sum of VC and RV), inspiratory capacity (IC: the sum of IRV and TV), functional residual capacity (FRC: the volume in the lungs at the end-expiratory position), vital capacity (VC: the volume of air breathed out after the deepest inhalation), maximal inspiratory pressure (MIP: the maximal pressure that can be produced by the patient trying to inhale through a blocked mouthpiece) and maximal expiratory pressure (MEP: the maximal pressure measured during forced expiration (with cheeks bulging) through a blocked mouthpiece after a full inhalation). Measuring pulmonary mechanics assesses the ability of the lungs to move large volumes of air quickly through the airways to identify airway obstruction.

Presence of Microbe causing Infection/Sickness/Mortality

The presence and amount of microbes that can cause infection, sickness, and/or mortality are key facility performance indicators.

Food Spoilage/Shelf Life

The rate of spoilage and shelf life of food products in a facility are key facility performance indicators for food processing and storage facilities. Examples include the shelf life of perishable foods in a wholesale or retail food facility.

Bioburden

The bioburden in food products (i.e., the colony forming units per gram of product) is a key facility performance indicator for food processing and storage facilities. The presence and amount of any known pathogen(s) or indicator taxa/OTUs in a food product is also a key facility performance indicator for these types of facilities. Bioburden can be measured by luminometer, which is a method that measures the total amount of adenosine triphosphate (ATP) in a sample. ATP is usually only present in living cells, so the amount of ATP in a sample is sometimes used as a general indicator of bioburden. Bioburden can also be measured in embodiments other than food products, such as the amount of bioburden in a carpet.

### Production Yield and Efficiency/Operational Continuity

The yield, efficiency, and cost of any production method are key facility performance indicators. Examples include how often machinery or the facility needs to be shut down for cleaning, and how many days per week/month/year of operation are lost to microbiome-related issues. Another example is whether a cruise ship has to terminate a cruise early due to contamination/illness. Methods of the invention can be used to increase the operational continuity of assets.

Frequency and Type of Reportable Incidents

The frequency and type of reportable incidents relating to the microbiome of a facility are key facility performance indicators. For example, the requirement and frequency at which government or other authorities need to be notified of reportable incidents (FDA notification for food borne illness, food recall, CDC and state and local authorities for hospital acquired infection, identification of pathogens in incoming water supply) are key performance indicators for pharmaceutical and medical device manufacturers as well as medical facilities of all types.

Data Collection and Correlation

In accordance with the present invention, a wide variety of non-microbiome data can be collected and correlated with the facility microbiome. This data may be analyzed and used to improve microbial conditions of the facility, thereby reducing and/or preventing performance reductions. Examples include type of ventilation, air flow, exposed surface composition (carpet, ceiling tiles, paint, upholstery, and fabric of staff clothing), lighting (natural and artificial), temperature, relative humidity, frequency of cleaning, chemicals used for cleaning, surface moisture pH, presence and amount of volatile organic compounds, formaldehyde, CO₂ level, O₂ level, CO level, NO₂ level, waste container location and frequency of removal, amount of airborne particulates and particle size distribution, lighting, facility volume, heating and cooling systems, and occupant density.

Historical Data

The present invention may further use historical data to monitor changes within the microbiome. For example, some implementations of the present invention analyze collected historical data from incidence of negative or positive facility performance indicators to identify and track changes in the microbiome. Such data might include, for example and without limitation, infection rate data, the time (of day, of year, of operation) a particular performance indicator occurred or was otherwise present, and facilities management data, such as when cleaning, servicing, or HVAC fluctuations have occurred, as well as temperature and relative humidity fluctuations.

Contemporaneous Data

The present state of performance indicators is also useful in correlating the facility microbiome and changes in the facility microbiome with facility performance indicators. This analysis is useful in identifying procedures and/or treatments such as changes in facility operation parameters that are most effective in improving the facility microbiome and therefore improving facility performance.

Correlating Data

The data collected regarding performance indicators is correlated with the facility microbiome and changes in the facility microbiome in accordance with certain aspects of the invention. This correlation can be within a given facility, across facility types, or across all facilities of a particular user. The correlation can be used in accordance with the invention to alter facility operation parameters in a way that increases (improves) facility performance.

Remediation Actions

Once a particular facility microbiome state or change in the facility microbiome is correlated with a facility performance indicator, the practitioner can, in accordance with certain embodiments of the invention, make changes to changeable facility operation parameters to increase the likelihood of favorable performance indicator conditions and/or reduce the likelihood of unfavorable performance indicator conditions. For example, actions that do not eliminate all microbes but rather allow microbes whose presence is associated with improved performance to remain but eliminate microbes whose presence is associated with decreased performance are an embodiment of the invention. Illustrative changeable facility operation parameters include the following.

HVAC

The HVAC system of a facility will often allow the manager of the facility to alter the temperature, humidity, air supply source, air flow and filtration of the air in the facility. Occupied spaces often ventilate at a rate of less than 1 air change per hour (ACH), and research shows that this is insufficient for diluting human-associated microbes in indoor air. Higher ventilation rates (e.g., 3 ACH) effectively remove airborne microbes emitted from human occupants, and introduce outdoor airborne microbes. Filtration in HVAC systems removes particulate matter from supply air sources. Office buildings often employ MERV-8 filtration, which remove most fungal spores, but not bacteria. Hospital operating rooms typically use more stringent filtration (MERV-15), which removes most bacteria from supply air. In some scenarios, such as operating rooms, more stringent filtering can improve performance, while in other buildings, such as offices surrounded by green space, unfiltered outdoor air would improve performance. An example of HVAC remediation is professional HVAC and duct cleaning. In many cases a building's operational parameters are set at a certain level and remain there regardless of changes to the outside air or facility performance, resulting in suboptimal performance over time.

For example, in times of high pollen and particulate matter, less outdoor air and more indoor recycled air may be advantageous because it reduces allergenicity and therefore lung function and other aspects of personal comfort and productivity. The present invention enables the operators of the facility to monitor these potential conditions and alter the environment to reduce the likelihood that air quality will cause employee health problems.

An additional consideration is energy use, and the present invention has many applications that can help improve energy efficiency. In some cases buildings are operated to use as little outdoor air as possible to save energy for heating and cooling; however, the effect this has on facility performance parameters such as hospital infection rates, lung performance of occupants, and employee absenteeism is not taken into account. As a general rule for building management at average occupant density levels of commercial office space, energy costs approximately $1 per square foot, rent costs approximately $10 per square foot, and employees cost approximately $100 per square foot. It is an object of the invention to maximize human performance through methods of the invention, which provides a better use of funds than simply minimizing energy use as a first priority for building management.

Cleaning Protocols and Frequency

The nature, frequency, and type of cleaning protocols are key changeable facility operation parameters. In particular the combination of location, frequency and identity of cleaning chemicals/reagents used is a key changeable facility operation parameter. Frequency and duration of sterilization using a device such as portable room disinfection systems that use pulsed xenon ultraviolet light to destroy viruses, bacteria, mold, fungus and bacterial spores in the patient environment that cause healthcare associated infections is a changeable facility operation parameter (see U.S. patent application Ser. Nos. 13/706,926 and 13/156,131, incorporated herein by reference).

Surface Alteration

The nature of exposed surface composition (carpet, paint, flooring, furniture upholstery, surgical gown fabric and lighting, including natural light) are key changeable facility operation parameters. Antimicrobial chemicals, such as triclosan, are commonly embedded in indoor materials, and can influence microbes on surfaces. Examples of surface alteration include replacing triclosan-embedded materials with copper or stainless steel surfaces, and replacing patient room carpets with non-porous linoleum flooring.

Facility Use and Design

Facility use and design, including room location (i.e., juxtaposition to other rooms and operations), ventilation duct routes, exposure of interstitial building spaces (i.e., duct work and water pipes in the ceiling), location of key functions that affect air quality (i.e., printers, 3D printers, computer servers), ventilation of food preparation spaces, window locations, window material choices, day-lighting strategies, and the movement of people and equipment through the facility are key changeable facility operation parameters. One example of facility use and design remediation afforded by the present invention is moving food preparation areas or other specially sensitive areas at least a certain distance (e.g., 30 feet) away from potential sources of undesired microbes (i.e., restroom doors). Another example is to reroute HVAC ventilation routes so that patient rooms exhaust directly to outdoor air or another designated location, i.e., a place where potential harmful microbes can be killed or otherwise rendered less harmful, instead of into an interior area, such as a hallway or another patient's room.

Computer System for Controlling Operation Parameters

In another aspect, all embodiments of the present invention are provided in computer-assisted format. Thus, the present invention provides computer systems capable of assisting in the characterizing, correlating, and altering aspects of the various embodiments of the present invention. Once a particular application is designated of interest, a computer system configured to monitor a facility microbiome and modify one or more operation parameters in response to changes in the facility microbiome can be provided to control all or various steps of the method. The computer system can include one or more sensors configured to obtain samples, one or more processing units configured to analyze the samples, and one or more control units configured to modify one or more operation parameters based on the analysis of the samples.

In some embodiments, a sensor and processing unit are combined into a single unit such that the unit can be employed to both obtain and analyze samples. In such cases, data generated from the analysis can be transmitted to a control unit (e.g., a central server) where the data can be correlated with data received from other sensors/units and/or used to determine whether one or more of the facility's operation parameters should be modified.

In other embodiments, a sensor may comprise a standalone unit that requires that samples be manually collected and provided to the processing unit. In such cases, the processing unit and/or the control unit can be connected to the sensor for purposes of controlling the operation of the sensor (e.g., to control when the sensor obtains a sample). However, in some embodiments, one or more sensors may be passive sensors that are not communicatively coupled to the processing unit or the control unit. Accordingly, a computer system in accordance with embodiments of the present invention may employ any number and type of sensor.

Sensors may be placed in any suitable location of a facility in accordance with the invention. Moreover, buildings already equipped with sensors can be readily assessed and controlled in accordance with the methods of the invention, e.g., a building can easily be retrofitted to take advantage of the various aspects and embodiments of the invention of most value to its owners and users. In either case, for example, a sensor may be placed outside of a building near an HVAC inlet to monitor outdoor air conditions and the microbiome at that location. A sensor may also be placed in a heavily occupied space such as an open office environment or nurse station. Sensors in such heavily occupied spaces can detect microbe-laden dust that is re-suspended by the occupants or bacteria-laden particles or microbes that are shed by the occupants. Sensors may also be placed near restroom doors to detect fecal-associated bacteria leaving the restroom. Sensors can also be placed in operating rooms to detect pathogens present therein. Sensors may also be placed in a patient room as a means to identify the patient's microbiome, i.e., to the extent the microbiome of the patient's room differs from that of other locations, including other patient's rooms and/or more common areas in the same building or elsewhere, practice of the present invention can provide one with meaningful insight on the patient's microbiome.

Regardless of whether a sensor directly or indirectly provides samples within the computer system, the control unit can collect data generated from the analysis of samples obtained from one or more sensors. In some embodiments, the control unit can provide a user interface (e.g., a webpage or mobile application) through which a user can view the collected data. For example, the control unit can provide a dashboard for accessing and exploring data that was collected over a specified period. The dashboard may provide a summary of the collected data such as, for example, a number of times during a particular period that a pathogen was detected in the collected data. The dashboard may also distinguish between collected data that was obtained from samples taken in one location of the facility and collected data that was obtain from samples taken in another location of the facility. For example, the dashboard may indicate the number of times that a pathogen was detected in any room or area of a building, e.g. an operating room of a hospital (i.e., the number of times the pathogen was detected in collected data that was based on samples obtained in the operating room).

In many embodiments of especially valuable applications of the invention, the control unit can also be configured to store collected data in association with one or more operation parameters that existed at the time the sample(s) on which the collected data is based were collected. In such cases, the control unit may be configured to obtain data representing the current operation parameters from the various systems of the facility (e.g., ventilation parameters from the HVAC system). For example, collected data that was generated based on one or more samples that were collected at a first time can be correlated with one or more operation parameters that existed at the first time. In this way, the user can better identify the effect that the one or more operation parameters may have had on the microbiome or predict it for future use. In similar fashion, this measurement and comparison may be repeated over time, and over multiple sites, to gain ever more sophisticated control of the BE microbiome of any facility of any industry of interest.

As an example, the control unit may store a first set of collected data in association with a first set of operation parameters, a second set of collected data in association with a second set of operation parameters, and a third set of collected data in association with a third set of operation parameters. All such data sets can be identified as to time of collection and compared with data sets taken at other times. The first and second sets of collected data may indicate that the microbiome included a harmful level of a particular microbe while the third set of collected data may indicate that the level of the particular microbe in the microbiome was no longer harmful. The user may then analyze the first, second, and third sets (and three of course is not the upper limit) of operation parameters to identify that a change occurred in the operation parameters between the second and third sets. The user could then conclude that the reduction in the level of the harmful microbe was likely a result of the change. Accordingly, by correlating collected data with operation parameters, the control unit can assist the user in identifying the effectiveness of changes in the operation parameters of a facility.

Also, this correlation of collected data with operation parameters can, in accordance with the invention, assist the user in identifying when a change in the operation parameters should be made such that the user makes the change as a result of the characterization provided. For example, when the user identifies from the collected data that the microbiome currently includes a harmful level of a microbe, the user can then review the current operation parameters to determine whether any change can and should be made to improve the microbiome. In such cases, the user interface can provide options for manually modifying one or more changeable operation parameters. For example, the user interface may include an option for modifying an HVAC operation parameter such as a ventilation parameter.

In some embodiments, the control unit is configured to process the collected data automatically to identify when a change to one or more operation parameters should be made. In such cases, the control unit can automatically effect the identified change or can prompt a user to confirm whether the change should be effected. In some embodiments, the control unit can be configured to implement a learning mode in which the control unit identifies the effect on the microbiome that one or more changes to the operation parameters has. For example, after any change is made to one or more operation parameters, the control unit may monitor the collected data to identify any changes in the microbiome. If the control unit determines that a change to a particular set of one or more operation parameters consistently results in a particular change in the microbiome, the control unit may update its configuration to automatically cause the change to the particular set of operation parameters whenever the current collected data indicates that the particular change in the microbiome would be appropriate.

Examples of Computer System Control of an HVAC System

In accordance with one or more embodiments of the present invention, a computer system provided by the invention is employed to monitor indoor and outdoor microbes and BE microbiomes as well as other biological indicators, and in response control one or more operation parameters of an HVAC system. For example, an outdoor sensor can be employed in accordance with the invention to monitor the allergenic fungal spore concentrations in the outdoor air near an HVAC inlet. The sensor may be configured to transmit data representing these concentrations to the control unit on a periodic basis. The control unit can be configured to cause the HVAC system to introduce an amount of outdoor air as long as the concentration of allergenic fungal spores is below a threshold, and to cause the HVAC system to stop introducing outdoor air once the concentration exceeds the threshold. Additionally, the control unit may be configured to cause the HVAC system to recirculate the indoor air through a high stringency filter (such as MERV-13 or higher) when the threshold is exceeded.

The control unit may be further configured to monitor the concentration of allergenic fungal spores or pollen after the change to the HVAC system has been effected. This monitoring can include monitoring the concentration in the indoor air (e.g., using a different sensor) to identify the effectiveness of the change to the HVAC system as well as monitoring the concentration in the outdoor air to determine when outdoor air can again be introduced.

As another example, a computer system may include a sensor that is positioned within an operating room to detect the MRSA pathogen. The sensor can be configured to transmit data to the control unit which indicates whether the MRSA pathogen is present in the air within the operating room. If the control unit determines that the received data indicates that the MRSA pathogen is present, the control unit can control the HVAC system to cause an increase in the amount of unfiltered outdoor air that is supplied to the operating room.

In summary, a computer system in accordance with embodiments of the present invention can be configured to monitor the microbiome of a facility and modify one or more operation parameters to address detected changes in the microbiome. This monitoring and modifying can be performed on a real-time basis to ensure that the microbiome remains acceptable.

Benefits of Remedial Action

A wide variety of benefits can be achieved by remedial action taken in accordance with the invention, including but not limited to decreased infection, sickness, and/or mortality rates; reduced operations costs, energy savings, increased production rate/yield of products manufactured in a facility, longer operational continuity of facilities or equipment in facilities, reduced employee sick leave, reduced cleaning requirement(s), reduced reportable incidents, increased exposure to beneficial microorganisms, reduction in antibiotic-resistance development, reduced likelihood of asthma and allergy triggers, and improved occupant comfort and satisfaction.

### EXAMPLES

### Example 1

### School

A study to analyze and adjust the microbiome of a school is performed in accordance with the present invention to improve the BE of the school. An occupied school building is selected to illustrate the influence of various facility parameters on the types and concentrations of airborne microbes (termed “the airborne microbiome”) within the school.

Active Air Sample Collection

Active air samples are collected as follows:_1) 10 active vacuum air samples (8 inside and 2 outside) are collected on each floor of the school each day. 2) Each air sample collection is commenced at 8 am and ends at 6 pm, for a total of 10 hours per air sample. 3) Each air sample consists of two 25 mm cellulose ester filters having 1.4 um pore diameter. 4) Air is drawn through each filter using a vacuum pump at a rate of 3 liters per minute, resulting in 1.8 m³ of air being passed through each filter. 5) Each air sample is collected after 6 pm, sealed, and frozen until laboratory processing.

Passive Air Sample Collection

Some of the active air sample locations further include passive air samples. Passive air samples are collected as follows: 1) Each passive air sample comprises a single, empty, sterile petri dish. 2) The empty, sterile petri dishes are exposed to the air by laying the lid and the base of the petri dish face up, side-by-side on a shelf that is affixed to a wall in proximity to the active air sample. 3) Airborne particles that settle into the passive air samples are collected as a biological sample. 4) Three different sampling durations are used, namely, 10 hours, 48 hours, and 168 hours. 5) Replicate passive air samples are obtained for the 10 hour and 48 hour sampling durations during the 168 hour sampling duration. 6) Following the respective sampling duration, each passive air sample is collected, sealed and frozen until laboratory processing.

Surface Sample Collection

Microbial communities on 220 surfaces are sampled throughout the school as follows: 1) Each surface sample is collected by wiping the surface sample area (approximately 20 cm²) with a sterile cotton swab. 2) Surface sample areas include: desks, chair seats, countertops, keyboards, light switches, door handles, walls, refrigerator handles, restroom stall doors, toilet seats, and sinks. 3) Each surface sample is sealed and frozen until laboratory processing.

Building Parameter Measurements

Building parameters of the school are measured throughout the study. The school's built-in environmental monitoring system measures a host of parameters, including: temperature, relative humidity, particulate matter, VOCs, carbon monoxide, carbon dioxide, and other indicators. Additional data is collected at each sampling site, including: temperature and relative humidity.

Bioinformatic Analysis

High throughput sequencing methods (such as Illumina MiSeq, Illumina HiSeq, or Roche 454 pyrosequencing) are used to generate DNA sequence files for each of the collected samples. The DNA sequence files undergo bioinformatics analysis entailing file manipulation, sequence transformations, quality filtering/control, and clustering of similar sequences into operational taxonomic units (OTUS). The results of the bioinformatic processing are summarized on an OTU table and analyzed along with building parameters as follows:

1. Co-occurring OTUs from air samples are grouped and correlated with ventilation treatments, occupancy patterns, and with environmental parameters such as temperature and humidity to detect patterns over time.

2. Groups of co-occurring OTUs are then analyzed for their ability to predict changes in environmental conditions. For example, were it is determined that human occupants in a building always result in increased Staphylococcus-related OTUs, and a decrease in Acinetobacter OTUs, this determination is used to focus analysis on important indicator OTUs.

3. OTU tables and sequence files are scanned for the presence of known allergens and pathogens, as well as genes that cause both distinctions, genes that produce toxins, or genes that otherwise influence human health. These tables and files are also compared with building parameters to generate actionable recommendations for reducing exposure to these factors.

4. OTUs from swab samples are correlated with a specific geographic positions within the school, surface type, and contact type. In some cases, surface cleaning regimens are evaluated in the presence of an OTU or group of OTUs to generate actionable recommendations for changes to the cleaning regimens.

Results

In accordance with the present invention, the bioinformatics analysis is used to create an indoor microbiome profile for the test facility. The microbiome profile is able to accurately i) determine and characterize the indoor environment of the test facility; ii) determine suitability for microbial survival and growth as well as dispersal potential from microbial sources; and iii) determine occupant load and behavior. Actionable information is derived from the statistical analysis, which results in recommendations for changes in the design, layout, and management of the school, as well as the behavior of the occupants. Illustrative recommendations include, without limitation, one or more of the following:

(i) Frequent detection of human fecal associated OUT's in the air adjacent to a restroom suggests that food preparation areas should be moved at least some minimal distance, e.g., 30 feet, away from restroom doors to avoid food contamination. Such a recommendation might result when sensors within 30 feet of the restroom door detect human fecal associated bacteria, but are not detected more than 30 feet from the restroom doors. If rearranging the location of the food preparation area of the school is not feasible, an alternate recommendation might be that restroom and office ventilation rates should be increased from 1 AHC to 3 AHC to avoid airborne movement of human fecal associated OTUs into food preparation areas. Such a recommendation could be based on results from altering ventilation rates that demonstrate that human fecal associated bacteria are not detected at the restroom doors of restrooms with higher ventilation rates.

(ii) Detection of food borne Salmonella bacteria on kitchen surfaces in the school indicate insufficient or inappropriate cleaning regimens.

(iii) Periodic testing of lung function of students and teachers, and correlation of lung function with airborne microbiome patterns and building operating parameters.

### Example 2

### Hospital

This example describes practice of the invention in a hospital.

Samples may be taken from all areas where typical (up to all) patients are located and from different places in those areas, including bedsheets, air, doorknobs, and equipment in rooms. Samples may be also taken from entry points, including carpet in main hallways and from air intake and ventilation system exit points in those areas. Samples may be taken from health care staff-associated items, including surgical gowns, surgery instruments, catheters, and doctors' and nurses' hands. Samples may be collected and analyzed on a weekly basis (in other examples, other sample frequencies are employed).

During the sampling period, readings are collected from sensors at various locations throughout the building measuring one or more of the following: temperature, air flow, and relative humidity are recorded, as are other HVAC parameters, for the hospital. In addition, the type, frequency, and location of all or certain types of infections are tracked, along with patient data (i.e., was the patient immunocompromised, what were the symptoms, what medications was the patient on, for what was the patient initially admitted, and what procedures did the patient undergo). The type, severity, outcome and frequency of infections is an illustrative performance indicator of the system for this example.

In one embodiment of this example, all microbial DNA in the samples (metagenomic, not just 16S, sequencing) taken from facility is sequenced, and the building parameters are correlated with performance indicators. In other embodiments, only sequencing of specific target sequences is performed.

Remedial action in the hospital focuses on those areas in the facility where a metagenomic or other match occurs between microbes that infected patients and locations where that microbe is present, particularly where it is proliferating. The remedial action taken includes cleaning with disinfectants, removal of porous fabric curtains from the patient rooms, and increased ventilation rates when patient rooms are occupied. Dissemination patterns are studied to evaluate whether remedial action is needed or could be beneficial in that regard.

In one embodiment, the sequencing occurs at the hospital so that remedial action can be taken in real-time or near real-time. For instance, real-time sequencing detects the presence of airborne MRSA in hallway sensors, activating the HVAC system to increase ventilation rates to 10 ACH, and exhaust hallway air directly to the outside of the building or through a UV sterilization unit, instead of recirculating exhaust air.

### Example 3

### Meat Processing Facility

In this example, a complex of 12 poultry processing facilities, including 3 that have consistently higher Salmonella counts per kilo of processed product leaving facility, is the subject of, location for, the practice of the invention.

Sampling occurs at various locations in each facility (walls, carpet, entries, and exits) and at any or all of various surfaces (including equipment that handles or otherwise is in contact with the meat). HVAC data for each facility is recorded over the sampling period, and a correlation of the building parameters in the top 25% of facilities with best performance (lowest Salmonella burden) and the bottom 25% of facilities (with worst performance) is made.

The correlation demonstrates that, for example, even though temperature is set at certain range for all facilities as a standard operating procedure, the relative humidity is higher in the worse performing facilities (i.e., because location is near a body of water). This may show, for example and without limitation, that relative humidity is more important than temperature, at least for some temperature ranges, at these facilities. The correlation may also show that certain meat suppliers to the facility have more contaminated product than others.

Remedial actions may include, for example and without limitation, adjustments to the HVAC system to control humidity in a desired range, adjustments to the cleaning protocol and frequency, and selecting different meat suppliers.

### Example 4

### Cruise Ship

Samples are taken from any of various locations all over the ship, particularly from common areas, such as dining facilities, game rooms, and meeting rooms, and from food-related areas, such as kitchens, food storage rooms, dishwashing rooms, and refrigerators. HVAC parameters are measured and recorded, and detailed records are made of the cleaning protocols and frequencies at all locations. Sequence analysis is performed as promptly as possible, in some embodiments on the ship itself, and remedial action is taken as promptly as possible. In some instances, in the presence of pathogenic samples (i.e., norovirus nucleic acid detected), remedial action is taken (area is disinfected) as soon as a potential problem is identified. In other instances, samples are collected and only analyzed and correlated with other data once an outbreak of some pathogen caused illness occurs, so that the correlations are used to guide future activities (i.e., use of a different cleaning protocol or different frequency of cleaning to reduce outbreaks on future trips). One example outcome of the last instance is the finding that the first detection of norovirus three days prior to the outbreak is in the cruise ship kitchen during preparation for initial departure. This suggests early and thorough sampling throughout the kitchen before every trip to develop an early detection system for future outbreaks.

### Example 5

### Commercial Building

This example illustrates various aspects and embodiments of the invention by demonstrating how the methods of the invention can be applied to evaluate the impact various alternative air filters can have on the indoor microbiome of an office building. This example, which was conducted in an actual facility, is reported here to be illustrative only, as the methods illustrated can be applied to any facility parameter, as described above, not simply the air filter. Accordingly, the nature and actual performance of the filters evaluated is provided merely to demonstrate that actual test data drove the analysis provided.

The facility was equipped with three levels of supply air filtration arranged in series: first, a MERV-13 HVAC filter (0.3-1 um particle filtration at 89-90% efficiency); second, a MERV-15 filter (94% efficiency for 0.3-1 um particles); and third, and a carbon filter. The filters could be, and were selectively removed, allowing for all three, zero, or any combination of the filters to be in place. Sampling was generally as follows. Four biological samples were collected from the input face on each of the three filter types, thus capturing microbial cells and other debris trapped by the filter. Each of the 12 biological samples was thus contained in a single sterile cotton swab that had been wiped across the filter surface such that all sides of the cotton swab were covered in dust substrate from the filter surface. These collected samples were sealed in sterile packaging and frozen on site. Samples were kept frozen (−80 C) until processing.

Whole genomic DNA was extracted from each sample using the PowerSoil DNA Isolation kit (MoBio, Inc. Carlsbad, Calif.), following manufacturer's instructions. All samples were processed for 16S and ITS2 amplicon sequencing, and one sample from each filter was also processed for whole genome shotgun (WGS) sequencing.

### Amplicon Sequencing

For this illustrative demonstration, the Internal Transcribed Spacer 2 (ITS2) region and the 16S rDNA V4 region were used for the analysis involving amplicon sequencing. Other sequences, as alternatives or in addition, could have been used.

The ITS2 region (of the ribosomal RNA operon) of any samples containing it was amplified by PCR and sequenced following a protocol adapted from published methods (see Human Microbiome Project, C. (2012), Structure, Function and Diversity of the Healthy Human Microbiome, Nature 486(7402): 207-214; and Human Microbiome Project, C. (2012), A Framework for Human Microbiome Research, Nature 486(7402): 215-221). The sequencing was done on the MiSeq platform (Illumina) using the 2×300 bp paired-end protocol (see Caporaso et al., Ultra-high-throughput microbial community analysis on the Illumina HiSeq and MiSeq Platforms, ISME Journal 2012; 6(8): 1621-4). Primers ITS3 and ITS4 (see White et al, Amplification and Direct Sequencing of Fungal Ribosomal RNA Genes for Phylogenetics, PCR Protocols: A Guide to Methods and Applications, Edited by Innis et al., NY: Academic Press Inc; 1990:315-322) containing adapters for MiSeq sequencing and 12mer molecular barcodes were used for amplification.

The 16S rDNA V4 region was also amplified by PCR and sequenced on the MiSeq platform, but a 2×250 bp paired-end protocol was used, yielding pair-end reads that should overlap almost completely. The primers used for amplification contain the gene primers (515F and 806R), adapters for MiSeq sequencing, and dual-index barcodes so that the PCR products can be pooled and sequenced directly (see Aporaso).

The final 16S and ITS libraries were sequenced on the Illumina MiSeq platform (300 PE). After sequencing, raw sequence files were processed using QIIME 1.9, and 97% similarity operational taxonomic units (OTUs) were assigned taxonomy with the GreenGenes bacterial database. All subsequent analysis was conducted in R.

Whole Genome Sequencing

Each whole genomic sample was sheared into fragments of approximately 500-600 base pairs using the E210 system (Covaris, Inc. Woburn, Mass.). Products were then amplified through Ligation Mediated-PCR (LM-PCR), performed using the HiFi DNA Polymerase (Kapa Biosystems, Inc., Cat. No. KM2602). Purification was performed with Agencourt AMPure XP beads after enzymatic reactions. Following the final XP bead purification, quantification and size distribution of the LM-PCR product was determined using the Agilent Bioanalyzer 7500 chip.

Libraries were pooled in equimolar amounts to achieve a final concentration of 10 nM. The library templates were prepared for sequencing using Illumina's cBot cluster generation system with TruSeq PE Cluster Generation kits. Briefly, this library was denatured with sodium hydroxide and diluted to 7 pM in hybridization buffer to achieve a load density of 756K clusters/mm². The library pool was loaded in a single lane of a HiSeq 2500 flow cell, which was spiked with 1% phiX control library for run quality control. The sample then underwent bridge amplification to form clonal clusters, followed by hybridization with the sequencing primer. Sequencing runs were performed in paired-end mode on the HiSeq 2500 platform. Assisted by the TruSeq SBS kits, sequencing-by-synthesis reactions were extended for 101 cycles from each end, with an additional 7 cycles for the index read. After sequencing, .bcl files were processed through analysis software (CASAVA, Illumina), which demultiplexes pooled samples and generates sequence reads and base-call confidence values (qualities). Resulting reads were mapped against the Antibiotic Resistance DataBase (ARDB). Reads that were closer than 80% identity cutoff with an E-Value less than 0.0001 were used to infer antibiotic-resistance potential. Gene functions that were more than 1% abundant, against the Kyoto Encyclopedia of Genes and Genomes (KEGG), were used to assemble metabolic pathways.

The filters contained significantly different microbial communities, indicating, as expected, that filter types and filter combinations are building operation parameters that can be modulated to alter the microbiome of the building, and thus alter facility performance for indicators such as infections, allergic reactions, lung function, antibiotic resistance, volatile organic compound production, volatile organic compound degradation, bacterial toxicity, fungal toxicity, bacterial sporulation, building material degradation and viral infectivity.

### Results: Antibiotic Resistance

The MERV-13 filter contained a significant number of antibiotic resistance activities that were not found on the other two filters, notably four different types of vancomycin resistance genes as well as genes imparting resistance to the crucial antibiotics streptomycin and gentamicin. The MERV-15 filter, which has a tighter stringency/smaller pore size, contained a small and distinct set of antibiotic resistance activities, including a set of activities not found on the other two filters. The carbon filter, which operates mainly for the purpose of removing organic molecules, contained a set of antibiotic resistance activities that were also distinct from the other two. Table 2 summarizes antibiotic resistance genes that were only discovered on one of the three filters, with corresponding mechanisms of action for the particular gene type. Some antibiotic resistance activities were discovered on two or all three filters as well.

TABLE 2
Antibiotic Resistance on the MERV-13 Filter

| Resistance Profile | Description |
|---|---|
| Antibiotic resistance found only on MERV-13 filter | |
| tobramycin, dibekacin, 6_n_netilmicin, gentamicin, | Aminoglycoside N-acetyltransferase, which modifies |
| netilmicin | aminoglycosides by acetylation. |
| butirosin, kanamycin, isepamicin, paromomycin, | Aminoglycoside O-phosphotransferase, which modifies |
| lividomycin, gentamincin_b, amikacin, neomycin, | aminoglycosides by phosphorylation. |
| ribostamycin | |
| butirosin, kanamycin, gentamicin_b, isepamicin, | Aminoglycoside O-phosphotransferase. |
| paromomycin, amikacin, neomycin, ribostamycin | |
| streptomycin | Aminoglycoside O-phosphotransferase. |
| penicillin, cephalosporin | Class A beta-lactamase, which opens the beta-lactam ring. |
| penicillin, cephalosporin, cephamycin, carbapenem | Class B beta-lactamase, which opens the beta-lactam ring. |
| penicillin, carbapenem, cephalosporin, cephamycin | Class B beta-lactamase, which opens the beta-lactam ring. |
| chloramphenicol, fluoroquinolone | Major facilitator superfamily transporter. Multidrug resistance efflux |
| | pump. |
| streptogramin_b, lincosamide, macrolide | ABC transporter system, Macrolide-Lincosamide-Streptogramin B |
| | efflux pump. |
| fluoroquinolone | Major facilitator superfamily transporter. |
| macrolide, lincosamide, streptogramin_b | rRNA adenine N-6-methyltransferase, which can methylate adenine |
| | at position 2058 of 23S rRNA. |
| tigecycline | Multi antimicrobial extrusion (MATE) efflux family protein. |
| | Multidrug resistance efflux pump. |
| tetracycline | Major facilitator superfamily transporter, tetracycline efflux pump. |
| streptomycin | Streptomycin resistance protein. |
| tetracycline | Xanthine-guanine phosphoribosyltransferase. Mechanism detail |
| | unknown. |
| tetracycline | Ribosomal protection protein, which protects ribosome from the |
| | translation inhibition of tetracycline. |
| Antibiotic resistance found only on MERV-15 filter | |
| netilmicin, dibekacin, amikacin, sisomicin, isepamicin, | Aminoglycoside N-acetyltransferase. |
| tobramycin | |
| penicillin, carbenicillin | Class A beta-lactamase, which opens the beta-lactam ring. |
| carbenicillin, penicillin | Class A beta-lactamase, which opens the beta-lactam ring. |
| chloramphenicol | Group A chloramphenicol acetyltransferase. |
| chloramphenicol | Major facilitator superfamily transporter, chloramphenicol efflux |
| | pump. |
| trimethoprim | Group A drug-insensitive dihydrofolate reductase. |
| lincomycin | ABC transporter system, Macrolide-Lincosamide-Streptogramin B |
| | efflux pump. |
| fluoroquinolone | Resistance-nodulation-cell division transporter system. Multidrug |
| | resistance efflux pump. |
| tetracycline | NADP-requiring oxidoreductase, an enzyme that can modify |
| | tetracycline. |
| Antibiotic resistance found only on carbon filter | |
| aminoglycoside, fluoramphenicol | Resistance-nodulation-cell division transporter system. Multidrug |
| | resistance efflux pump. |
| acriflavine, aminoglycoside, macrolide | Resistance-nodulation-cell division transporter system. |
| cephalosporin | Class C beta-lactamase, which opens the beta-lactam ring. |
| penicillin | Class A beta-lactamase, which opens the beta-lactam ring. |
| n_cephalosporin, monobactam, e_cephalosporin, penicillin | Class A beta-lactamase, which opens the beta-lactam ring. |
| cephalosporin | Class A beta-lactamase, which opens the beta-lactam ring. |
| macrolide, streptogramin_b, lincosamide | rRNA adenine N-6-methyltransferase. |
| lincosamide, streptogramin_b, macrolide | rRNA adenine N-6-methyltransferase. |
| macrolide, lincosamide, streptogramin_b | rRNA adenine N-6-methyltransferase. |
| chloramphenicol | Major facilitator superfamily transporter. |
| chloramphenicol, acriflavine, norfloxacin | Major facilitator superfamily transporter. |
| puromycin, acriflavine, t_chloride | Resistance-nodulation-cell division transporter system. |
| fluoroquinolone | Pentapeptide repeat family, which protects DNA gyrase from the |
| | inhibition of quinolones. |
| streptogramin_b, lincosamide, macrolide | ABC transporter system, Macrolide-Lincosamide-Streptogramin B |
| | efflux pump. |
| tetracycline | Ribosomal protection protein. |
| tetracycline | Major facilitator superfamily transporter, tetracycline efflux pump. |
| thiostrepton | Specifically methylates the adenosine-1067 in 23S ribosomal RNA. |
| | Confers resistance to antibiotic thiostrepton. |

These results demonstrate that microbiome analysis, integrated with data on building operation parameters, can be used to determine which types of a target biochemical activity are entering a building (e.g. antibiotic resistance), which sub-types are present (e.g., tetracycline resistance), and which building operation parameters can be changed to alter the microbiome of the building (e.g., removal of particular filter or bypassing a filter during periods when outside air meets certain requirements, such as having an upper limit on pollen, other particulates, or the presence of a certain predetermined nucleic acid consensus sequence). Thus, these results illustrate that the microbiome of a building can be characterized, controlled, and altered using the methods of the invention and without actually identifying a particular type of harmful or beneficial microbe is present but instead by amplifying entire genomes and simply assessing how much of it is from organisms that harbor genes associated with an undesirable microbe.

### Results: Metabolic Pathways

The MERV-13 filter contained a significant number of metabolic pathway activities that were not found on the other two filters. The MERV-15 filter, which has a tighter stringency/smaller pore size, also contained a set of activities not found on the other two filters. The carbon filter also contained a set of activities that were also distinct from the other two. Table 3 summarizes metabolic pathway activities that were only discovered on one of the three filters. Some metabolic pathway activities were detected on two or all three filters as well.

TABLE 3
Filter Activity

| Activities found only on | Activities found only on | Activities found only on carbon |
|---|---|---|
| MERV-13 filter | MERV-15 filter | filter |
| Histidine transport system | Type IV secretion system | V-type ATPase, prokaryotes |
| GINS complex | | Ascorbate biosynthesis, animals, |
| | | glucose-1P => ascorbate |
| Rhamnose transport system | | Complex II (succinate |
| | | dehydrogenase/fumarate |
| | | reductase), fumarate reductase |
| AI-2 transport system | | DNA polymerase epsilon complex |
| Ergocalciferol biosynthesis | | Reductive citric acid cycle (Arnon- |
| | | Buchanan cycle) |
| DNA polymerase delta complex | | Fatty acid biosynthesis, initiation |
| GABA biosynthesis, prokaryotes, | | Triacylglycerol biosynthesis |
| putrescine => GABA | | |
| N-glycan precursor biosynthesis | | V-type ATPase, prokaryotes |
| Oligosaccharyltransferase | | |
| Reductive pentose phosphate cycle | | |
| (Calvin cycle) | | |
| Reductive pentose phosphate cycle, | | |
| glyceraldehyde-3P => RuBP | | |
| Lignin biosynthesis, cinnamate => | | |
| lignin | | |
| Lysine biosynthesis, 2-oxoglutarate => | | |
| 2-aminoadipate => lysine | | |
| DNA polymerase III complex, | | |
| bacteria | | |
| Capsaicin biosynthesis, L- | | |
| Phenylalanine => Capsaicin | | |
| Cholesterol biosynthesis, FPP => | | |
| cholesterol | | |
| Ascorbate biosynthesis, plants, | | |
| glucose-6P => ascorbate | | |
| Sec (secretion) system | | |
| C10-C20 isoprenoid biosynthesis, | | |
| plants | | |
| Spliceosome, U2-snRNP | | |
| Sphingosine biosynthesis | | |
| Holo-TFIIH complex | | |
| Ceramide biosynthesis | | |
| GPI-anchor biosynthesis, core | | |
| oligosaccharide | | |
| Spliceosome, U1-snRNP | | |
| Spliceosome, 35S U5-snRNP | | |
| Castasterone biosynthesis, | | |
| campesterol => castasterone | | |
| Origin recognition complex | | |
| Histidine transport system | | |
| GINS complex | | |
| Rhamnose transport system | | |
| AI-2 transport system | | |
| Ergocalciferol biosynthesis | | |
| DNA polymerase delta complex | | |
| GABA biosynthesis, prokaryotes, | | |
| putrescine => GABA | | |
| N-glycan precursor biosynthesis | | |
| Oligosaccharyltransferase | | |
| Reductive pentose phosphate cycle | | |
| (Calvin cycle) | | |
| Reductive pentose phosphate cycle, | | |
| glyceraldehyde-3P => RuBP | | |
| Lignin biosynthesis, cinnamate => | | |
| lignin | | |

Results: Reduction in Number and Diversity of OTUs

Filter 1 Drastically Reduces Both the Number and the Diversity of Bacterial OTUs.

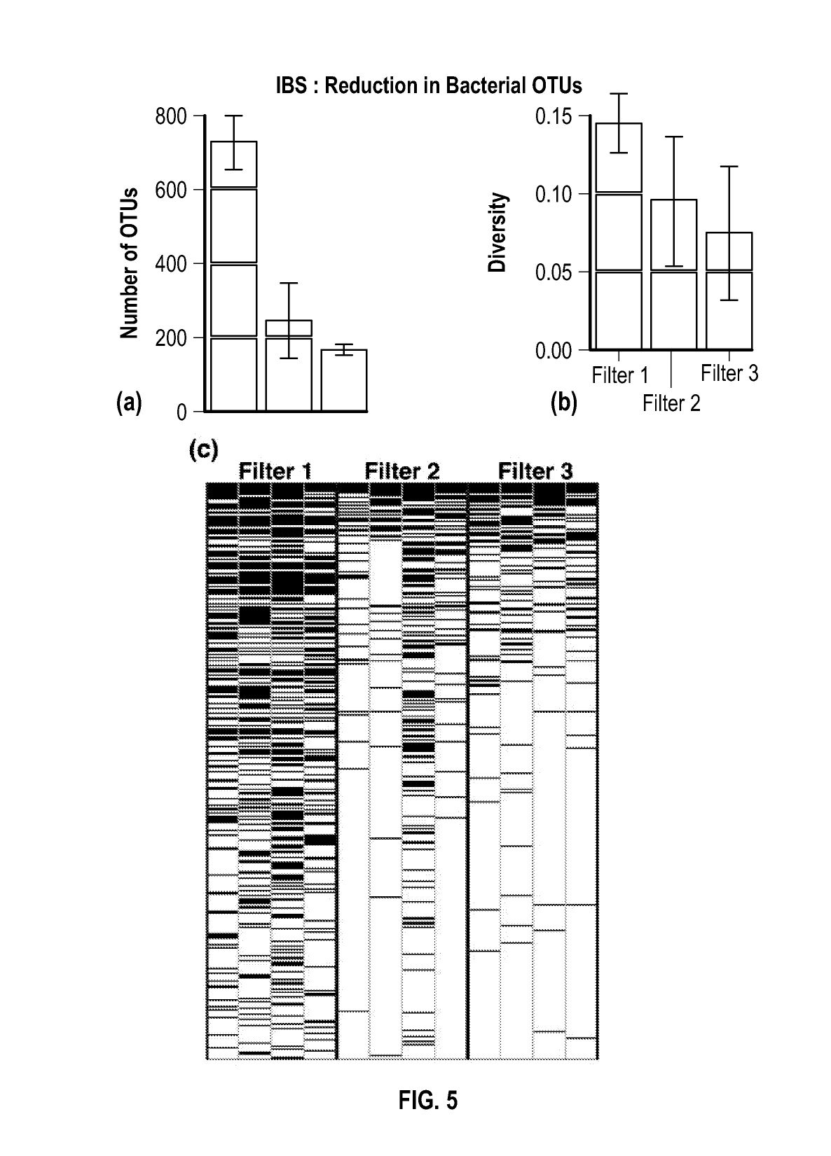

With reference to FIG. 5, taxonomic diversity is a combined metric embodying both species richness and the relative distributions of taxa. Part (a) of FIG. 1 shows the total number of bacterial OTUs that was reduced by passing air through Filter 1. The taxonomic diversity of bacterial OTUs that was reduced by passing air through Filter 1 is shown in part (b). Error bars representing standard errors for the 4 samples for each filter are shown in part (c), wherein each horizontal band demonstrates the presence (black) or absence of a bacterial genus found on each filter. Bands are shown (top to bottom) in order of their total abundance.

Filter 1 Drastically Reduces Both the Number and the Diversity of Fungal OTUs.

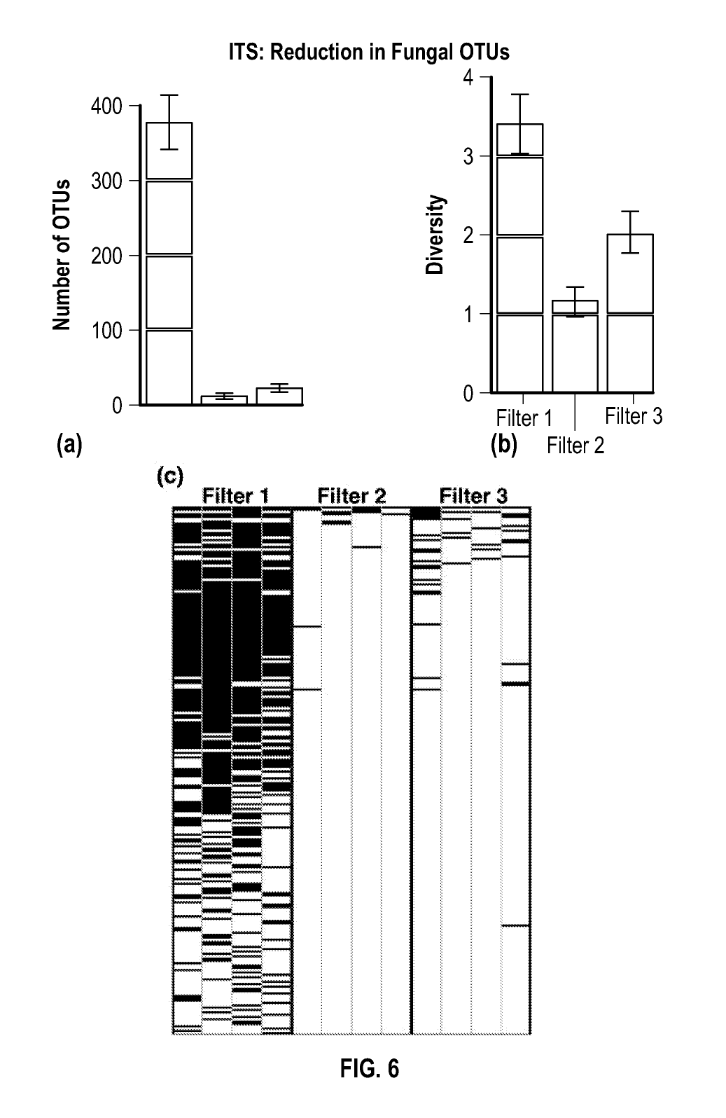

With reference to FIG. 6, part (a) shows the total number of fungal OTUs that was reduced by passing air through Filter 1. The taxonomic diversity of fungal OTUs that was reduced by passing air through Filter 1 is shown in part (b). Error bars representing standard errors for the 4 samples for each filter are shown in part (c), wherein each horizontal band demonstrates the presence (black) or absence of a fungal genus found on each filter. Bands are shown (top to bottom) in order of their total abundance.

Filter 1 Drastically Reduces Both the Number and the Diversity of Pollen-Related OTUs, as Well as their Abundance.

With reference to FIG. 6, different levels of pollen were found on the three filters. The first filter, a MERV-13, captured the largest number of different types of pollen, as well as the highest level of diversity of pollen.

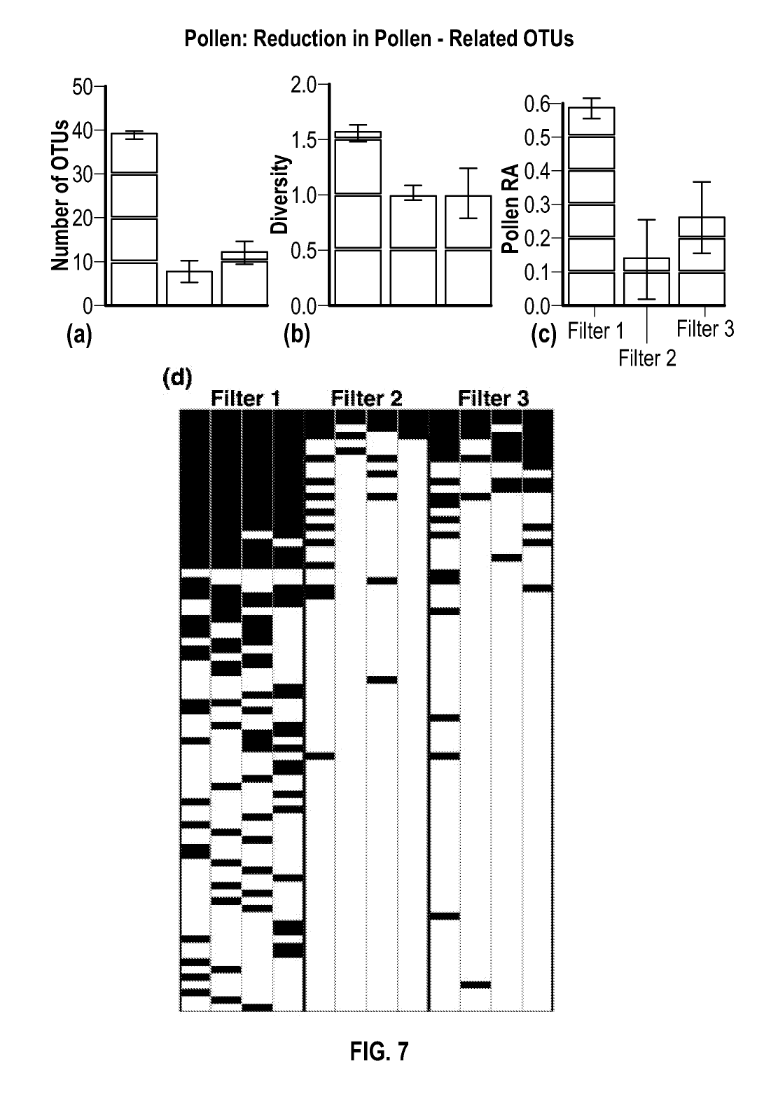

Part (a) of FIG. 7 shows the total number of plant pollen OTUs that was reduced by passing air through Filter 1. The taxonomic diversity of plant pollen OTUs that was reduced by passing air through Filter 1 is shown in part (b). The total relative abundance (RA) of pollen-related DNA sequences was also reduced after air passed through Filter 1, as shown in part (c), which further includes error bars representing standard errors for the 4 samples for each filter. Each column of part (d) shows a single filter sample, wherein each horizontal band demonstrates the presence (black) or absence of a pollen-associated OTU found on each filter. Bands are shown (top to bottom) in order of their total abundance.

As demonstrated by the results shown in FIGS. 5-7, Filter 1 was successful in reducing microbial diversity, and also pollen diversity and abundance. This example illustrates with actual data how the methods of the invention can be used to identify an operation parameter that can be altered to achieve a desired state in the BE and so is merely illustrative of the broad application of the instant invention.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method for improving the performance of a facility, said method comprising (i) correlating a facility microbiome with one or more facility operation parameters to identify changes in the facility microbiome that contribute positively or negatively to a facility performance indicator; (ii) identifying changes in the microbiome that correlate with facility operation parameters, wherein said changes can be prevented or caused by altering a changeable facility condition; and (iii) altering one or more changeable facility conditions to effectuate the desired change in one or more facility performance indicators.

2. The method of claim 1, wherein the one or more alterations to the changeable facility conditions comprises an alteration that preferentially induces or reduces proliferation or dissemination of one or more microbes, biological activities, or operational taxonomic units over another.

3. The method of claim 2, wherein the one or more alterations preferentially induce proliferation or dissemination of one or more microbes, biological activities, or operational taxonomic units distinct from those of the group consisting of Streptococcus pneumonia, Klebsiella, Staphylococcus aureus, Candida albicans, Pseudomonas aeruginosa, Acinetobacter baumannii, Stenotrophomonas maltophilia, E. coli O157:H7, Clostridium difficile, Mycobacterium tuberculosis, Enterococcus, Legionella pneumophila and Streptococcus pyogenes.

4. The method of claim 1, wherein the one or more alterations to the changeable facility conditions comprises an alteration that preferentially induces or reduces proliferation or dissemination of biochemical activities that are correlated with facility performance, the biochemical activities being measured using the presence and relative abundance of DNA or RNA molecules that impart the activity.

5. The method of claim 1, wherein the one or more alterations to changeable facility conditions comprises an alteration that preferentially reduces viability or proliferation of one or more microbes, biological activities, or operational taxonomic units.

6. The method of claim 5, wherein the one or more alterations preferentially reduces viability or proliferation of one or more microbes, biological activities, or operational taxonomic units of the group consisting of species or genera of Streptococcus pneumonia, Klebsiella, Staphylococcus aureus, Candida albicans, Pseudomonas aeruginosa, Acinetobacter baumannii, Stenotrophomonas maltophilia, E. coli O157:H7, Clostridium difficile, Mycobacterium tuberculosis, Enterococcus, Legionella pneumophila and Streptococcus pyogenes.

7. The method of claim 1, wherein the one or more alterations to changeable facility conditions comprises an alteration that reduces dissemination of one or more microbes, biological activities, or operational taxonomic units within the facility.

8. The method of claim 1, wherein the one or more alterations to changeable facility conditions comprises an alteration that reduces virulence of one or more one or more microbes, biological activities, or operational taxonomic units.

9. The method of claim 8, wherein the one or more alterations reduces virulence of one or more microbes, biological activities, or operational taxonomic units of the group consisting of Streptococcus pneumonia, Klebsiella, Staphylococcus aureus, Candida albicans, Pseudomonas aeruginosa, Acinetobacter baumannii, Stenotrophomonas maltophilia, E. coli O157:H7, Clostridium difficile, Mycobacterium tuberculosis, Enterococcus, Legionella pneumophila and Streptococcus pyogenes.

10. The method of claim 1, wherein changes in the facility microbiome are detected by nucleic acid sequencing of microbial DNA in samples taken from said facility, and said sequencing occurs simultaneously or within 15 minutes (real time) or within 15 minutes to within one day (near real-time), and said altering step occurs within 15 minutes to within one day of said sequencing.

11. The method of claim 10, wherein said sequencing is metagenomic.

12. The method of claim 1, wherein data for said facility operation parameters are displayed on a computer screen together with information characterizing said facility microbiome or changes therein, and information relating to facility performance indicators.

13. A method of modulating one or more target biochemical activities, microbes, or OTUs within a built environment facility, comprising: (a) collecting one or more samples from locations in said facility; (ii) subjecting the samples to DNA sequence analysis; (iii) quantifying the target biochemical activity by determining the number of sequences that fall within a predetermined sequence identity definition characterizing said biochemical activity(ies), microbes, or OTUs; and (iv) modifying at least one facility operation parameter that alters the number of sequences in the facility that fall within the predetermined sequence identity definition in a subsequent sampling.

14. The method of claim 13, further comprising a step of correlating a facility performance parameter with the facility operation parameter modification.

15. The method of claim 13, wherein the target biochemical activity is selected from the group consisting of allergenicity, antibiotic resistance, volatile organic compound production, volatile organic compound degradation, bacterial toxicity, fungal toxicity, bacterial sporulation, building material degradation and viral infectivity.

16. The method of claim 13, wherein the facility operation parameter involves the ventilation system of the facility.

17. The method of claim 16, wherein the facility performance parameter is lung function of occupants.

18. The method of claim 15, wherein the target biochemical activity is antibiotic resistance, and the activity is resistance to one or more of the following antibiotics: 6_n_netilmicin, acriflavin, acriflavine, amikacinaminoglycoside, apramycin, astromicin, bacitracin, beta_lactam, butirosin, carbapenem, carbenicillin, cefoxitin, ceftazidime, ceftriaxone, cephalosporin, cephamycin, chloramphenicol, ciprofloxacin, cloxacillin, deoxycholate, dibekacin, doxorubicin, e_cephalosporin, enoxacin, erythromycin, fluoramphenicol, fluoroquinolone, fosfomycin, fosmidomycin, gentamicin, glycylcycline, isepamicin, imipenem, meropenem, kanamycin, kasugamycin, lincomycin, lincosamide, lividomycin, macrolide, methicillin, monobactam, n_cephalosporin, neomycin, netilmicin, norfloxacin, paromomycin, penicillin, polymyxin, puromycin, ribostamycin, roxithromycin, sisomicin, spectinomycin, streptogramin, streptomycin, sulfonamide, t_chloride, teicoplanin, tetracenomycin, tetracycline, thiostrepton, tigecycline, tobramycin, trimethoprim, vancomycin, erythromycin, clindamycin, doxycycline and minocycline, and the facility performance parameter is selected from the group consisting of number, frequency and/or outcome of infections of patients or occupants, and health or growth of animals.

19. An automated facility system comprising:
a. means for collecting and sequencing microbiome samples from the facility;
b. means for measuring facility operation parameters; and
c. means for automated modification of facility operation parameters in response to detection of nucleotide sequences that fall within a predetermined sequence identity definition;
wherein the facility operation parameters are modified to optimize facility performance on an ongoing basis as sequence data is obtained from the samples.

20. The facility system of claim 19 that is capable of performing a method of claim 1.

21. The facility system of claim 19, wherein an automated modification occurs through a system that prioritizes human or animal health over minimizing energy use and effects facility operation through changing (a) ventilation flow rates and/or (b) the ratio of indoor:outdoor air entering the HVAC system and/or (c) the amount and type of air filtration.

22. A method of optimizing the bioburden of a surface material in a facility comprising: (a) placing two or more distinct surface materials in an identical location within a facility or a test chamber, (b) measuring one or more facility operation parameters, (c) analyzing the microbiome of the materials, and (d) determining which materials harbor an identity and/or relative abundance of microbes and/or OTUs that are associated with improved facility performance compared to others.

23. The method of claim 22, wherein said facility is a hospital, an office building, a food preparer or a food processor, or a seaborne or airborne vessel.

24. A system for improving facility performance, comprising:
multiple collectors positioned at various locations within a facility, said collectors configured to collect samples potentially containing nucleic acid;
a nucleic acid sequencer operably connected to the collectors and configured to sequence any nucleic acid therein so as to determine whether one or more indicator taxa, biochemical activity, or OTU is present in the sample and to send a signal if such nucleic acid is detected in an amount predetermined to generate the signal;
a control unit operably coupled to one or more devices of the facility, the one or more devices performing a function that is related to an operational parameter of the facility, the control unit further being operably coupled to receive a signal from the nucleic acid sequencer, the control unit comprising computer executable software for performing a step for receiving a signal that the one or more indicator taxa, biochemical activity, or OTU is present in a sample in an amount that requires adjusting a setting of the one or more devices to alter the level of the nucleic acid detected after one or more operational parameters have been changed.

25. The system of claim 24, wherein the nucleic acid sequencer sequences the one or more samples in real-time or near real-time.

26. The system of claim 24, wherein the step for receiving a signal that the one or more indicator taxa, biochemical activity, or OTU is present in a sample in an amount that requires adjusting a setting the one or more devices to alter the level of the nucleic acid detected is performed in time increments form every 15 minutes to weekly.

27. The system of claim 24, wherein the facility is a hospital.

28. The system of claim 24, wherein the facility is an office.

29. The system of claim 24, wherein the facility is a cruise ship.

30. The system of claim 24, wherein the facility is an airliner.

31. The system of claim 24, further comprising a user interface for viewing at least one of i) the sequence of the one or more indicator taxa, biochemical activity, or OTU, ii) the level of nucleic acid detected, and iii) the setting of the one or more devices.

32. The system of claim 24, wherein the one or more indicator taxa, biochemical activity, or OTU is selected from the group consisting of a pollen, a fungus, a virus, or a bacteria.

33. The system of claim 24, wherein the level of nucleic acid detected indicates the presence of one or more microbes.

34. The system of claim 24, wherein the one or more devices is an HVAC system, and the step of adjusting the setting of the HVAC system comprises adjusting (a) ventilation flow rates and/or (b) the ratio of indoor:outdoor air entering the HVAC systems and/or (c) the amount and type of air filtration.

35. The system of claim 24, wherein the step for receiving a signal that the one or more indicator taxa, biochemical activity, or OTU is present in a sample in an amount that requires adjusting a setting the one or more devices to alter the level of the nucleic acid detected is performed at intervals determined by occupancy within the facility.

36. The system of claim 24, wherein the step for receiving a signal that the one or more indicator taxa, biochemical activity, or OTU is present in a sample in an amount that requires adjusting a setting the one or more devices to alter the level of the nucleic acid detected is performed at intervals determined by one or more environmental considerations selected from the group consisting of a season, a time of the day, a day of the week, a proximity of the facility to a source of one or more indicator taxa, and a weather event.

37. The system of claim 24, wherein level of nucleic acid detected is altered to a lower level.

38. The system of claim 24, wherein the level of nucleic acid detected is altered to a higher level.

39. The system of claim 24, wherein the operational parameter is selected from the group consisting of air flow, exposed surface composition, lighting, temperature, relative humidity, frequency of cleaning, chemicals used for cleaning, surface moisture pH, CO2 level, O2 level, NO2 level, waste container location and frequency of removal, amount of airborne particulates and particle size distribution, facility volume, heating and cooling systems, human occupancy patterns, occupant traffic patterns, and occupant diversity.

40. A method for improving facility performance, comprising:
collecting from a facility a sample potentially containing nucleic acid;
sequencing any nucleic acid present within the sample so as to determine whether one or more indicator taxa, biochemical activity, or OTU is present in the sample;
adjusting one or more operational parameters of the facility to alter the level of the nucleic acid detected.

41. The method of claim 40, further comprising a step for providing multiple collectors at various locations within the facility, said collectors configured to collect a plurality of samples potentially containing nucleic acid.

42. The method of claim 40, further comprising a step for adjusting a setting of one or more devices of the facility, the devices performing a function that is related to the one or more operational parameters of the facility, wherein the step of adjusting the setting of the one or more devices alters the level of the nucleic acid detected.

43. The system of claim 24, wherein the step for receiving a signal that the one or more indicator taxa, biochemical activity or OTU is present in a sample in at least a minimum predetermined amount activates a warning devices that audibly and/or visibly indicates that (a) an action should be taken and/or (b) an indicator taxa, biochemical activity, or OTU is present.


SEQUENCE LISTING (6 sequences)

SEQ ID NO: 1 - DNA, 2471 bp, Staphylococcus aureus
aaatttttatcttcaattgcatcaatagtattattaatttctttatctttggaagcataaaaatatataccaaacccgacaactacaactattaaaataagtggaacaatttttatctttttcatcaatatcctccttatataagactacatttgtagtatattacaaatgtagtatttatgtcaaaataatgttataatttttgtgatatggaggtgtagaaggtgttatcatcttttttaatgttaagtataatcagttcattgctcacgatatgtgtaatttttttagtgagaatgctctatataaaatatactcaaaatattatgtcacataagatttggttattagtgctcgtctccacgttaattccattaataccattttacaaaatatcgaattttacattttcaaaagatatgatgaatcgaaatgtatctgacacgacttcttcggttagtcatatgttagatggtcaacaatcatctgttacgaaagacttagcaattaatgttaatcagtttgagacctcaaatataacgtatatgattcttttgatatgggtatttggtagtttgttgtgcttattttatatgattaaggcattccgacaaattgatgttattaaaagttcgtcattggaatcgtcatatcttaatgaacgacttaaagtatgtcaaagtaagatgcagttctacaaaaagcatataacaattagttatagttcaaacattgataatccgatggtatttggtttagtgaaatcccaaattgtactaccaactgtcgtagtcgaaaccatgaatgacaaagaaattgaatatattattctacatgaactatcacatgtgaaaagtcatgacttaatattcaaccagctttatgttgtttttaaaatgatattctggtttaatcctgcactatatataagtaaaacaatgatggacaatgactgtgaaaaagtatgtgatagaaacgttttaaaaattttgaatcgccatgaacatatacgttatggtgaatcgatattaaaatgctctattttaaaatctcagcacataaataatgtggcagcacaatatttactaggttttaattcaaatattaaagaacgtgttaagtatattgcactttatgattcaatgcctaaacctaatcgaaacaagcgtattgttgcgtatattgtatgtagtatatcgcttttaatacaagcaccgttactatctgcacatgttcaacaagacaaatatgaaacaaatgtatcatataaaaaattaaatcaactagctccgtatttcaaaggatttgatggaagttttgtgctttataatgaacgggagcaagcttattctatttataatgaaccagaaagtaaacaacgatattcacctaattctacttacaaaatttatttagcgttaatggcattcgaccaaaatttactctcattaaatcatactgaacaacaatgggataaacatcaatatccatttaaagaatggaaccaagatcaaaatttaaattcttcaatgaaatattcagtaaattggtattacgaaaatttaaacaaacatttaagacaagatgaggttaaatcttatttagatctaattgaatatggtaatgaagaaatatcagggaatgaaaattattggaatgaatcttcattaaaaatttctgcaatagaacaggttaatttgttgaaaaatatgaaacaacataacatgcattttgataataaggctattgaaaaagttgaaaatagtatgactttgaaacaaaaagatacttataaatatgtaggtaaaactggaacaggaatcgtgaatcacaaagaagcaaatggatggttcgtaggttatgttgaaacgaaagataatacgtattattttgctacacatttaaaaggcgaagacaatgcgaatggcgaaaaagcacaacaaatttctgagcgtattttaaaagaaatggagttaatataatggataataaaacgtatgaaatatcatctgcagaatgggaagttatgaatatcatttggatgaaaaaatatgcaagtgcgaataatataatagaagaaatacaaatgcaaaaggactggagtccaaaaaccattcgtacacttataacgagattgtataaaaagggatttatagatcgtaaaaaagacaataaaatttttcaatattactctcttgtagaagaaagtgatataaaatataaaacatctaaaaactttatcaataaagtatacaaaggcggtttcaattcacttgtcttaaactttgtagaaaaagaagatctatcacaagatgaaatagaagaattgagaaatatattgaataaaaaataaaattgttgtgtttacaacaatacatagaaaacagaggaaacaatcaagtcgttgaatatttcctctgttttttagttgaaaaaattaaccgaaagcctgaatgcaagtcttgattaaa

SEQ ID NO: 2 - DNA, 4378 bp, Tyzzerella nexilis
acccaagtccttggcgatgttgcctacgaaggagcctctgtcgatctcttcccgcacagaatagcgaatctgcccggccctggcctcccacagggtcgccaaaaggaagcacagcaggaccagctttctgcagtgtggcaacttttgcagagccgccatttccctcctgtcgcaattttctccgagtcaacttccagcatccagtgcggaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgccctttgtgtcaggttcaccgccagacacggtcacatcaccattggctgtggatttccaagaagcaaaggagccaatctcagcaaagctcgcactggcatttttagctgcttaaatttgaagagcagttcagcaaagcttgtgctcccttctagtcctataggtggcaggtgctgtggagctggcacagagtggtagacgaggaagcaggccagcatg

SEQ ID NO: 3 - DNA, 9041 bp, Burkholderia pseudomallei
cgccgccgccgggccgctcgggcgcgcccatcgcggttcgccggctcgcgtttcgccgcgcggaccgcttcgcgatcgagccgatgtcgttcgatctgccggcgggcagctacacggcgatcgtcgggcacaacggctcgggcaagtcgacgctcgcgaagatcatcgcggggcggctggcgcccgacgaaggcgtcgtcacgtatggcgacgtcgatctgtaccgcgtcgcgagcgacgcgcgccaccgcttcgcgctatacgtgccgcaggacgtcgcgctgctgaaccgctcgctgcgggagaacgtgcgttactacccgtcgacgctcaccgacgacgacgcggcccggctgctcgagcgcctcgcgtttcacaaggacggccggcccatcgatctcgacggcgaagtcggcgaaggcggcgcgcgcctgtcgggcggccaggtgcagaaggtcgagctcgtcaggctgatgggcgtcgacgttccggcgatcgtgctcgacgaaaccacgtcggggctcgatccgcacagcgatgcgctcggcatcgcgatgctgcgcgagcgcctcggccagcgcaccacgctcgtgctgatcacccaccggatcgcgaacgtcgaggcggccgaccaggtgctgttcctgtcgggcgggcggctcgtcgcggcggggccgcaccggcgcctcgtcgacacctgcgacgaataccgggcgttctggcgccgccagcccgaaccggccgacgcgaaggcgtgatgcgcgcgcggcgcgcgcgccgttgcagcgcgcacgcgggcgaacccggttcagtagatgaacagccgaatcgtcgccgtcccggtgaacggcccgatcttcggcgcgccgaacgacttcagcaccgcatcgagcacgatgttctgatacgacacgttggccggcatctgcgtgagcacgaactgccggttgaaggcaatcgcgtggccgttcctgtcctcgatgccgatcccgaagttgctgtcgctctgcggcacgagcaggccgtcgcgcaccgtctgcgacgtctcgaagtagccgtcgacgcgcaccgcgatgctgcatttcttcgtcaacgcgaggctgaacggcttgcgcggcaccgccggcgaaaagccgttgccggacgactggacctgcccgaacttgacgatccccggctcgggcgtgaccgtcacgtcgaccatgcacggcgtcggcttcaggttctgcagcccgctcagcttgtactggaagctcgggttcgtgttgttcaggcccttctcgccgtcgaactggaacaccgtgtagacatcgcccggcggctgcacccacgcgcccttcttgcgcacgaccacctggtaggtgatgctgaccggaatcttcgggcagcgcttcgcgttgaagtccgtctgcgagcacggcggcaccgcgaagcccgtcggcacaccggtgccggggcggctgcccgcgccgaaatggtcgatgccccgatagcggatgccgatctcgagcccccacgcggccggattcttgccgtccggattcgcgtagaagtagatgttgtcgacgatgttcaggttgcccgcgccgccgagatccttgtagcaatagccgtccgtcgtgcgcggcgcggaaatccagatcacgtagccgtccggcgcgtcggtcggatacgacgcgacgttgccgatcggctcggtgagcgaggtctggccgctgttcgtcaggcagcgcaccgcccacgcgggctgcgcgagcgcgacgacaagcgccgcgcacgcccatgcgagccagcccggcgcgcgccgcggcaaaagacgggcgagcgcgctcatcgcttcacgctgcggcacgcgaggccgtcgcacgtgtattcgaccgtcacctggccgccgtagtcgtcgacgtgcgtgacgaacaggccgcccgccgccgacgccgagaacggcacgctcgccgcgctcttcggattcaccatcaccggatcgagcggcagcgccgcccggttcgcgcccgctgcgagcgcgacgaccgtcacgtggtacggcgtcgggttgtcgaacacgagcttgcgcgcggccgcatcgacgcgcagcgtcatcggcagcgtcgggtcctcgtcgcgcgcgggctgcacgcccgccggccggtagaacagcttcatctgcgtgtgcagcgcgatttgcagcgcgttcggcgtgtccgtcttcggcggcacctcgcggatgttcagatagaacaccgattcgcggtcggcgggcagcgtcgcgcccggcatccgcgcgatgcgaagcacgttgcgctccttcggctcgacgcgctgcagcggcggcacgaccatcagcggcgtcgtgatccggttgccgcgctcgtcctcgagccacgactgcacgagatacggatacgcatcgctcttgttcgagagcgtcacgatcgcggcctgctcgccttcgttgaggatcacgcgcgtgcggtcgggcacgatcgccgcgtgcgcggcgcccgccagcgcgatgaagagcgcggcggccgcgcgccgtgcgcgcgccgcgcacgggaatcgatgggtcatgtcggtcatcggatcgggtcggtcggtggttcggtggttcggtggttaggtcggttaggtcggttaggtcggttaggtcggttaggtcggttaggtcggttaggtcggttaggtcgggtttgaattcgaggggcgggacggcgcgccgcgctcagcgcgacgcgtgctgcgcggcggcttcgcaggccaccggcgcgggcgtgccgtcgagctgcagcgtgtcgggaagcgcggcgggcgtgcagatcgtcttcgcgccggcgcgcacgacgagcttcgcgcgcggctgcacctgcgtcaggtacgcggcgcccgcctcgccgacgatgccgagctccttgccggtcgcggcgtcctgcaccgacgcgccgaacggcaccggcgcgccgccgtcgcccgtcagcgtgacgaacacgttgcgcccgcgcgccgcgttgaaccggatgtagccgatcgcgccgtcggtgagcacggtgcgctgaatcgggttcgtcacgtcggtgtcgaggccgagcttctcgacgctcacggtcgcgtcgtacacgttgtacggcgacacgccgtcgatcaccgcatagccgcgcgcattcgtgcgggcgtacgagccggacagcggcacgccgggcacgccgtcggtcgacacgagcaggcgcgtgtcgcccgcgttgccgttcgcgtgcgcggtcacgccgtagcgcgtcgcgacgaacgagccgtcgacctcgagcgacgccgacgcgtacgcgttcgcgaccgtcgacgcctgcgcggtgagctggtacgacggcgtgcgctgccgcaagctcgcgttcgccgacgcgcggccgtcggtcgtgccgccgtacagctgataggtgcgcccgttcgcgccatcgtagaggtagcccgcgttcacgctcgtgccgccctcgcccgccgatacgctcgacgtgagcgtctggcgctcgccgagcggcagcgtcgccgtcagcgacacctggttgccgccgccgcccgcgccttgcgtgcggaacgcggagaaacccaggttcacgctcttcagcgcgccgagcgagaacgcgcgcgtgagcgtcacgccgatgcgccgatcggacgggcgcgcccagtaggtcgtctggtcgtacgagaaataggtcgacgtatcgccgaagcgcttggagagcatcgccgaatagcgctgcttgccgttcgcgaggcccgacgcggtcgggtcgcccgagaactgctggaaattcgtgtagtcgcgctcggagaagcgatagccgaagaagcgcacgtcggcgtcgagcgcgtcgacgtgcttcgaatagttgattcgatacgagttgccgcgcttcgtccgcccgttccaccagagcgtcgcgcgcgcgtgcgtcacgtcggccgacagcgcgccgaaacggccgaaatcgcggccgacgccgaacgcgaccgacgtatagccggacgccgcgatgaggccgccgtagacggtcacgtcgaacggcagcccgtacgcggcctcggcgaagccgaaccacggcgtgatgcccgcgccgccgaaggtgcgcggctggccgatcgcggtcttgtagcgcagttggccctcgcgcgcgagaaacggcaccgccgcggtcgcgacctggaagcgctgcaccgtgccgtcctcctcctcgacgacgacgtcgagcgtgccctgcacgctcgtgttcaggttcgacagcgcgaacgcgcccggcgacaccttcgtcatgtacagcacgcggctgtcctgcatcaccttgacgatcgcgttggtgcgcgcgacgcccgtgacgagcggcgcgtagccgcgcatcgaaggcggcagcatccggtcgtcgctcttcatcgcgacgcccgacatcgagaacgtgctgaagatgtccgaatcgacgtagatctcgccgaacgacagcgtcgagcgaatcgacggcagcgcgcgatacgcgtacagctggttgaagcggaacgcgcgctccgcgtacacggcgccgcccgcgcgagtctgcgcctgataatcgccgcgaaagcgccacgcgccccagttcgcgccgatcgtgccgtacgcctgcaccgcattgttctgctgcgcgccgcggccgaacgcgtggttcgcgttcgcgagcacgcgatagtcgagcatcgcgccgtcgacgccgtcgctccagcgctcgggcggcaggtacgacgcatcggcgaattcgagcgcggcttgcgcaatcgtgatcttcaggcgcccttcgcctttctgataacggatcgtcacgccgtcgagcgacgcgagatccgcgcagcggccgcccatcgtgcgcggcaggttctcgacgagcgacttcttcagcccgaactgcgcgacgagctcgggcgcgacgcacgcgcgcgcgccctgcccgtcgtcgtgcgcgacgaattcgatcggctgcaggccgaagaacacgtcgttgacctgcacgtcgagcaggtatgtgcccggcagcgtgtagtccgcctgcgcgaactgcgacaggtcgacgtcgttcctgccgtcgatgctcaggaacgacgcattgaactccgtcgcccgggcatggctgccggcggccagcatgaacacgcagaggaaggaatgtctgattcgcacgccgactgactgaaaccgtaacgggatggggatcgcggaaaaagcgggaacgcgaagcggattcgcgcgaggaaaaggcgcgcgccctttcctcgcggtgcggctcgtcgagccgcgccggcttactggtagcgaaccgtgaagttcgcgacgccgtcggccgtgcctggcgtcacgctcgccgcggtcgattcgtacatcgcggcgaagcgcgcgacgctcgtgccgttgctcagcacgaccgggcctttctgctccgcgccgttgtcgaggtattcgcccgacgccgattgcagacggatgccgacgttcgtcgccgagccgacggtggcgatcagcttcggctgcgtcgcgttcgacgtgcccgtgaacgtgaagtacgcgttcttcgcgacgctcgtgtcgcagtcgacgagcttgatgtcgaagttctgcggcgtcgacttgtcgcccgccgccttgaacgtgttcgcggggacgaagccgaggcgcacggtctggtccaccgagcccgcgtcgatgccgcacgcgccggcgacgatctcgcccgtgaagttgagggtgccggtgccggcggcgaatgcggacgtggacaacgcggcgcaggcgacggcggacaggagagcttttttcatggctggttccgtaacgtgactaatgggattgggacgatttgccggacgtattcgcatcgcacgcgagaaatgctttgcaatgcattggtcttgattcgtcgcgacggaccgcattatcaagatgattcagaaagtattttgtaaatcattgtcagaaaatgacctttcggacggatttcggcgtgtgggatcaaatatttcgagcgaggcattttgcccaaggcggcgcgcttttccggtttttggggcatctcgacaagccattgtttttattatatttaatcgtttgcgcaatctcgcccgcgccgccgcaccgggcgggctgcggtgcgtcccgacccgccgcaccggcggccccgaatctgacatttcagcaagggttgtcaagtcgcaacatctcacacccccctcaatgtattttgatacaacaatctcattaaataaattcaatgcaatcgtttgcgctaaacgatcatcttataaaacgtcattctttcatttttcgcaagcatctgagcattcacgaatttgaatttatttgacaatttaaacgcaaattcatatttattgtgcaagtgaattccgaaatgaatgaaaccgcaatcgtttgcacgagtgcgaatcggtacggcgaaacgtttgcttgcccgctcacgaaaaatggtccgctgaatgcggccgcgcggcggcctcgcccgccgctcgagccggaccgctgcgatgaacgcgggaaggcgcgcgtgaagcgtcgcggcgttgcatcgagccgatccggccgcgtcaagcaagcgtcattcgcgcgcgcctcggagggtggaagcaaggcgagccacgcgcgcgacgccgtgacggccgccggcacacgggcgcggcgtgccgccccgaatcgcatacggcacgcgacggcaggatgcgtctgtccatcgcggagatcgaacgggcacaggcgctcagtcggcgcgacgcggcttgccggaccaccggtcactcgccccgctcatgcgccgcggcaacgtcacagcggacggcgggcacgatgcggcgcgctattcgatgggagggctcgcggaatcggcatgtgatggagagcgggcgccgtgggacgtcgttgtcgttgtcgttgtcgttgtcgttgtcgttgtcgcccgtcgagcggcaagcggcaagcggtccgccaggatcgagcgggttccactgtgcgcgacgcatcggtcaacacatggaatcgctccagacgacgggaaccgggatcgcgttggctgcatgaacgcagtgacgcgcgcgtggagcttcgtcacagcggcggtgacgatacgcgaatagccgcgtcacacagcggccgcgggcgggcgaaggtttaggtctgcatcgccgcgagcgcctactcggccgagctggcggcgcatcgatgccgatgtgtggccggccgccggcgaacgcggagcgggcgggaaatacgccttgacgcactcgctagggcgcttacgcgacacgaatcgtcgatcgcgcggcgaaaccggggcgcggcccctgatgcgcggccgcgcgctcgacgaggcggcgacgcgtgcgtgatgaatcatcccggagcgcgcgccgcgttcgttcggcgcgcgctccgcgatcgcattgatcgcatcagtcgcattggtcgcgacggcaactcaacacatcgacgccacgcgcgacggttccaatccgcacccggcacggcagcgccgacgaatggccgtgtgtacggcatcgttctgccgcgccgttatttcatgcgaacgaacaggaacggcaacacgccatagcggtcagcaaaagacgtgcatcgatatcgcacgaggcacgaggcacgaggcacgaggcacgaggcacgaggcacgaggcacgaggcacgaggcacagcgcgctcaagcgctcaagcgctcaagcacacccatcgccgccgcacccggcgcgcgaccagcccccgcccagcgcgcgatacagcgtgatcgcgttcgtgagccgcagctgcttcaggcgaatcagctcctgcccggactcgaacgtgctgcgctgcgcgtcgagcagctcgagatagctcgccacgccgctgtcgtagcgccgctgcgcgagccgcagccgctcggcgtccgcaccgtagaccgcctgctgcgcggcaagctgcgcgtcgatctgatcgcgcgccgcgagcgcatcggcgacttcgcgaaacgccgtctggatcgtcttctcgtattcggcgaccgcgatatgcttgcgcgcgtccgccacgtcgagattcgcacgattgcgcccgcccgcgaaaatcggcagcgtgagccgcggcgcgaacgtccacacgctcgagccggccgagaagagtcccgagaacgcgtcgctcaccgagccgacgtcggtcgtgagcgcgatgcgcgggaagaacgccgcgcgcgccgcgccgatgttcgcgttcgccgcgacgaggcgctgctcggcctgccggatgtccgggcgctgctcgagcagatccgacgacagccccggcgacacccgcgccaccgcgagcgcgtcgagcgcgggcgcatcggcgggcagcgccgtcatgaagtcgcccgcgagcagcttcagcgcgctcgccgcctgcgtgtgctcgcgctcgagcgccgccttcgacgcgcgcgccgacgcgacgagcatctcggccgtgcgcagctcgatcgccgtgctcgtgccggccgcgtaacggcgctgcgtgagcgcgtaggccgcgtcgcgcgcggcgagcgtgcgttcggcgagccccagttgatcgacgagcgcgcgctcggtcacgtacgcggacgcgacttcggcgatcaggctgatgcgcgccgcgcgctgcgcttcggcggtcgcgaagtactcggcgagcgccgcgtccgagaggctcttcacgcggccgaacagatcgatctcgaacgcgctcacgccgacactcgcgcgatacagcgagctcgtcgcgctctcgcgcagcaccgggtcgtaaagccgcgtgcgctcgtagccgaggctgccgtcgatcgacggcagccggtccgcgcgcgcgacgccgtagaggccgcgcgcttcctggatacgcagcgtcgcgatccgcaggtcgcgattgttcgcgagcgccgcgtcgatcagcgcgcgcagcgccggatcggtgaaatacgcgcgccagtcgtcgagccgcgcgtcgcgggcgtcgcgcccgg

SEQ ID NO: 4 - DNA, 664 bp, Pseudomonas aeruginosa
ctaccgcagcagagtctttgccagatttaaaaattgaaaagcttgatgaaggcgtttatgttcatacttcgtttaaagaagttaacgggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacga

SEQ ID NO: 5 - DNA, 3067 bp, Klebsiella pneumoniae
gatgtttgatgttatggagcagcaacgatgttacgcagcagggcagtcgccctaaaacaaagttaggccgcatggacacaacgcaggtcacattgatacacaaaattctagctgcggcagatgagcgaaatctgccgctctggatcggtgggggctgggcgatcgatgcacggctagggcgtgtaacacgcaagcacgatgatattgatctgacgtttcccggcgagaggcgcggcgagctcgaggcaatagttgaaatgctcggcgggcgcgtcatggaggagttggactatggattcttagcggagatcggggatgagttacttgactgcgaacctgcttggtgggcagacgaagcgtatgaaatcgcggaggctccgcagggctcgtgcccagaggcggctgagggcgtcatcgccgggcggccagtccgttgtaacagctgggaggcgatcatctgggattacttttactatgccgatgaagtaccaccagtggactggcctacaaagcacatagagtcctacaggctcgcatgcacctcactcggggcggaaaaggttgaggtcttgcgtgccgctttcaggtcgcgatatgcggcctaacaattcgtccaagccgacgccgcttcgcggcgcggcttaactcaggtgttagacggcaagaaaaggttccacgaactctgatgaaaaactactttgacagccctttcaaaggggagcttctttctgagcaagtgaaaaatccaaacatcaaagtaggccgttatagctattactctggctactatcacggccactcatttgatgaatgcgcgcgatacttgcatccagatcgtgatgacgttgataaattgatcattggcagcttttgttctataggaagcggggcttccttcatcatggctggcaatcaggggcatcggcatgactgggcatcatccttccccttcttctatatgcaagaggaacctgctttctcaagcgcactcgatgccttccaaagagcaggtgataccgccattggcaatgatgtctggataggctcggaggcaatgattatgcccggaatcaaaattggagacggtgccgtgataggtagtcgctcgttggtgacaaaagatgtagtgccttatgccatcatcggaggaagtcccgcaaagcaaattaagaagcgcttctccgatgaggaaatctcattgctcatggagatggagtggtggaactggccactggataaaattaagacagcaatgcctctgctgtgctcgtcaaatatttttggtctgcataagtattggcgcgagtttgtcgtctaacaattcattcaagccgacgccgcttcgcggcacggcttaattctggcgttagccaccaagaaggtgccatgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcaaccagaatatcagtggtggcattgacaaattctggttggaaggccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcccgcgcagatcagttggaagaatttgttcactacgtgaaaggcgagatcaccaaggtagtcggcaaataatgtctaacaattcgttcaagccgacgccgcttcgcggcgcggcttaactcaagcgttagatgcactaagcac

SEQ ID NO: 6 - DNA, 1823 bp, Acinetobacter baumannii
gttatggagcagcaacgatgttacgcagcagggcagtcgccctaaaacaaagttagccatatgaactcggaatcagtacgcatttatctcgttgctgcgatgggagccaatcgggttattggcaatggtcctaatatcccctggaaaattccgggtgagcagaagatttttcgcagactcactgagggaaaagtcgttgtcatggggcgaaagacctttgagtctatcggcaagcctctaccgaaccgtcacacattggtaatctcacgccaagctaactaccgcgccactggctgcgtagttgtttcaacgctgtcgcacgctatcgctttggcatccgaactcggcaatgaactctacgtcgcgggcggagctgagatatacactctggcactacctcacgcccacggcgtgtttctatctgaggtacatcaaaccttcgagggtgacgccttcttcccaatgctcaacgaaacagaattcgagcttgtctcaaccgaaaccattcaagctgtaattccgtacacccactccgtttatgcgcgtcgaaacggctaaccattccgtcaacgggacgccaaaatgctgcgcattttggttccctccgctgcgctccggctctcgttacgtccaacgttagcaccactgaaacccagctttatttagctcatgtttattcaaacggcatttagcttttcaggcgttattcagtgcctgttttgccttttttccgggcttcgcctgcatgggctgcgcaggttttcagtctttttggcctctagcccttgcgtagcaagcgcaagcagctatcgtttttgcagtgctgtgccgcctcggtggcgcagcgttttttcacggttagcgcccgtcgccaaattcaagttatccgttttggcttctggttctaacatttcggtcaagccgacccgcattctgcggtcggcttacctcgcccgttagacatcatgagggaagcggtgaccatcgaaatttcgaaccaactatcagaggtgctaagcgtcattgagcgccatctggaatcaacgttgctggccgtgcatttgtacggctccgcagtggatggcggcctgaagccatacagcgatattgatttgttggttactgtggccgtaaagcttgatgaaacgacgcggcgagcattgctcaatgatcttatggaggcttcggctttccctggcgagagcgagacgctccgcgctatagaagtcacccttgtcgtgcatgacgacatcatcccgtggcgttatccggctaagcgcgagctgcaatttggagaatggcagcgcaatgacattcttgcgggtatcttcgagccagccatgatcgacattgatctagctatcctgcttacaaaagcaagagaacatagcgttgccttggtaggtccggcagcggaggaattctttgacccggttcctgaacaggatctattcgaggcgctgagggaaaccttgaagctatggaactcgcagcccgactgggccggcgatgagcgaaatgtagtgcttacgttgtcccgcatttggtacagcgcaataaccggcaaaatcgcgccgaaggatgtcgctgccgactgggcaataaaacgcctacctgcccagtatcagcccgtcttacttgaagctaagcaagcttatctgggacaaaaagaagatcacttggcctcacgcgcagatcacttggaagaatttattcgctttgtgaaaggcgagatcatcaagtcagttggtaaatgatgtctaacaattcgttcaagccgaccgcgctacgcgcggcggcttaactccggcgttagatgcact